SEQUENCE LISTING (2 sequences)

SEQ ID NO: 1 - protein, 7 aa, Artificial Sequence
  Synthetic
YEVDGWK

SEQ ID NO: 2 - protein, 12 aa, Artificial Sequence
  Synthetic
HLNILSTLWKYR

(19) United States
(12) Patent Application Publication
(10) Pub. No.: US 2014/0303123 A1
(43) Pub. Date: Oct. 9, 2014

(54) SYNTHESIZING FUNCTIONALIZED DENDRIMERS WITHIN BIOLOGICAL SETTINGS

(71) Applicant: The Regents of the University of Michigan, Ann Arbor, MI (US)

(72) Inventors: James R. Baker, JR., Ann Arbor, MI (US); Sascha Nilu Goonewardena, Ann Arbor, MI (US); Hong Zong, Ann Arbor, MI (US)

(21) Appl. No.: 14/358,948

(22) PCT Filed: Nov. 15, 2012
(86) PCT No.: PCT/US2012/065252

§ 371 Date: May 16, 2014

Related U.S. Application Data
(60) Provisional application No. 61/562,767, filed on Nov. 22, 2011.

(51) Int. Cl. A61K 31/655; G01N 33/50

(52) U.S. Cl. CPC A61K 31/655; G01N 33/502
USPC 514/151; 435/375; 435/29

(57) ABSTRACT

The present invention relates to compositions (e.g., dendrimer scaffolds) capable of click chemistry for use in synthesis of functionalized dendrimers within biological settings, and methods of use of the same.

### STATEMENT REGARDING FEDERALLY SPONSORED RESEARCH OR DEVELOPMENT

This invention was made with government support under W911NF-07-1-0437 awarded by the U. S. Army Research Office. The government has certain rights in the invention.

### FIELD OF THE INVENTION

The present invention relates to compositions (e.g., dendrimer scaffolds) capable of click chemistry for use in synthesis of functionalized dendrimers within biological settings, and methods of use of the same.

### BACKGROUND OF THE INVENTION

The use of nanoparticle systems to monitor and modulate biological systems continues to grow. Their small size and dynamic properties are some of the features that endow nanoparticles with their unique capabilities. However, these same properties complicate their tracking and how they interact with biological systems. Typically, nanoparticles are labeled with fluorescent or radioactive handles so that their behavior can be monitored in biological systems. Although such labels have provided insight, these reporters can be difficult to incorporate into nanoparticles and may not faithfully represent their non-labeled counterparts. A method for direct monitoring nanoparticle systems without costly or time consuming synthetic protocols would further our understanding of the dynamic nanoparticle—biological interface without complicated synthetic schemes.

### SUMMARY OF THE INVENTION

The present invention is not limited to utilizing a particular type or form of dendrimer. Indeed, examples of dendrimers finding use in the present invention include, but are not limited to, PAMAM dendrimer, a Baker-Huang PAMAM dendrimer (see, e.g., U.S. Provisional Patent Application No. 61/251,244 and International Patent Application No. PCT/US2010/051835; each herein incorporated by reference in its entirety), a polypropylamine (POPAM) dendrimer, and a PAMAM-POPAM dendrimer. The type of dendrimer used is not limited by the generation number of the dendrimer. Dendrimer molecules may be generation 0, generation 1, generation 2, generation 3, generation 4, generation 5, generation 6, generation 7, or higher than generation 7. In some embodiments, half-generation dendrimers may be used. In certain embodiments, a generation 5 amine-terminated PAMAM dendrimer is used. In certain embodiments, a generation 5 alkyne-terminated PAMAM dendrimer is used. In some embodiments, the dendrimer is at least partially acetylated.

Dendrimers are not limited by their method of synthesis. The dendrimer may be synthesized by divergent synthesis methods or convergent synthesis methods.

In certain embodiments of the present invention, dendrimer molecules may be modified. Modifications may include but are not limited to the addition of amine-blocking groups (e.g., acetyl groups), ligands, functional groups, conjugates, and/or linkers not originally present on the dendrimer. Modification may be partial or complete. In some embodiments, all of the termini of the dendrimer molecules are modified. In some embodiments, not all of the dendrimer molecules are modified. In preferred embodiments, methods and systems of the present invention permit identification and isolation of subpopulations of dendrimers with known numbers of ligand attachments (e.g., conjugations) per dendrimer molecule, thereby yielding samples or subpopulations of dendrimer compositions with high structural uniformity.

‘Click chemistry’ involves, for example, the coupling of two different moieties (e.g., a therapeutic agent and a functional group) (e.g., a first functional group and a second functional group) via a 1,3-dipolar cycloaddition reaction between an alkyne moiety (or equivalent thereof) on the surface of the first moiety and an azide moiety (or equivalent thereof) (or any active end group such as, for example, a primary amine end group, a hydroxyl end group, a carboxylic acid end group, a thiol end group, etc.) on the second moiety. ‘Click chemistry’ is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932; each herein incorporated by reference in their entireties).

In certain embodiments, the present invention relates to compositions (e.g., dendrimer scaffolds) capable of click chemistry for use in synthesis of functionalized dendrimers within biological settings, and methods of use of the same. In particular, the present invention relates to conjugating alkyne-derivatized functional groups (e.g., ligands) via copper catalyzed 1, 3 dipolar cycloaddition reaction with azide-derivatized dendrimer nanoparticles within biological settings (e.g., administering an alkyne-derivatized functional group to a biological setting already having an azide-derivatized dendrimer such that, via click chemistry, the functional group conjugates with the dendrimer within the biological setting).

In some embodiments, the azide-derivatized dendrimer has no functional groups. In some embodiments, the azide-derivatized dendrimer has thereon one or more functional groups. In some embodiments, such functional group(s) are attached with the dendrimer via a linker. The present invention is not limited to a particular type or kind of linker. In some embodiments, the linker comprises a spacer comprising between 1 and 8 straight or branched carbon chains. In some embodiments, the straight or branched carbon chains are unsubstituted. In some embodiments, the straight or branched carbon chains are substituted with alkyls.

For example, in some embodiments, following introduction of azide-derivatized dendrimers conjugated with therapeutic agents and/or targeting agents into a biological setting (e.g., a cell sample), alkyne-derivatized imaging agents are introduced for the purpose of monitoring/tracking the location of the dendrimer within the biological setting. Such techniques permit, for example, assimilation of the dendrimer (e.g., functionalized or non-functionalized) into a biological setting prior to conjugation of the functional group.

The present invention is not limited to particular azide moieties (or equivalents thereof). In some embodiments, the azide moiety comprises the formula N₃⁻.

The present invention is not limited to particular alkyne moieties (or equivalents thereof). In some embodiments, the alkyne moiety is a cyclooctyne moiety (or equivalents thereof). In some embodiments, the cyclooctyne moiety comprises the following formula:

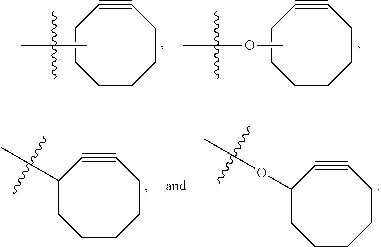

The present invention is not limited to a particular manner of conjugating the azide moieties (or equivalents thereof) and/or the alkyne moieties (or equivalents thereof) with either a dendrimer structure and/or a functional group. In some embodiments, the azide moieties (or equivalents thereof) and/or the alkyne moieties (or equivalents thereof) are conjugated with either a dendrimer and/or a functional group via a primary amine end group, a hydroxyl end group, a carboxylic acid end group, a thiol end group, etc.).

The present invention is not limited to a particular type and/or kind of biological setting for conjugating alkyne-derivatized functional groups with azide-derivatized dendrimer nanoparticles via copper catalyzed 1, 3 dipolar cycloaddition reactions. In some embodiments, the biological setting is either in vitro, in situ, or in vivo). Biological samples may be obtained from animals (including humans) and encompass fluids, solids, tissues, and gases. Biological samples include blood products, such as plasma, serum and the like.

The present invention is not limited to particular ligand types (e.g., functional groups). Examples of ligand types include but are not limited to therapeutic agents, targeting agents, trigger agents, and imaging agents. In some embodiments, the ligand is an azide ligand that includes an azido group. In some embodiments, the ligand includes an aromatic group. Methods, systems, and compositions of the present invention are not limited by the number of different ligand types used. There may be 1, 2, 3, 4, 5, 6, 7, 8, 9, 10 or more different types of ligands attached to a dendrimer molecule.

Examples of therapeutic agents include, but are not limited to, a chemotherapeutic agent, an anti-oncogenic agent, an anti-angiogenic agent, a tumor suppressor agent, an anti-microbial agent, an expression construct comprising a nucleic acid encoding a therapeutic protein, a pain relief agent, a pain relief agent antagonist, an agent designed to treat an inflammatory disorder, an agent designed to treat an autoimmune disorder, an agent designed to treat inflammatory bowel disease, and an agent designed to treat inflammatory pelvic disease. In some embodiments, the agent designed to treat an inflammatory disorder includes, but is not limited to, an antirheumatic drug, a biological agent, a nonsteroidal anti-inflammatory drug, an analgesic, an immunomodulator, a glucocorticoid, a TNF-α inhibitor, an IL-1 inhibitor, and a metalloprotease inhibitor. In some embodiments, the antirheumatic drug includes, but is not limited to, leflunomide, methotrexate, sulfasalazine, and hydroxychloroquine. Examples of biological agents include, but are not limited to, rituximab, finfliximab, etanercept, adalimumab, and golimumab. In some embodiments, the nonsteroidal anti-inflammatory drug includes, but is not limited to, ibuprofen, celecoxib, ketoprofen, naproxen, piroxicam, and diclofenac. In some embodiments, the analgesic includes, but is not limited to, acetaminophen, and tramadol. In some embodiments, the immunomodulator includes but is not limited to anakinra, and abatacept. In some embodiments, the glucocorticoid includes, but is not limited to, prednisone, and methylprednisone. In some embodiments, the TNF-α inhibitor includes but is not limited to adalimumab, certolizumab pegol, etanercept, golimumab, and infliximab. In some embodiments, the autoimmune disorder and/or inflammatory disorder includes, but is not limited to, arthritis, psoriasis, lupus erythematosus, Crohn's disease, and sarcoidosis. In some embodiments, examples of arthritis include, but are not limited to, osteoarthritis, rheumatoid arthritis, septic arthritis, gout and pseudo-gout, juvenile idiopathic arthritis, psoriatic arthritis, Still's disease, and ankylosing spondylitis.

Ligands suitable for use in certain method embodiments of the present invention are not limited to a particular type or kind of targeting agent. In some embodiments, the targeting agent is configured to target the composition to cancer cells. In some embodiments, the targeting agent comprises FA. In some embodiments, the targeting agent binds a receptor selected from the group consisting of CFTR, EGFR, estrogen receptor, FGR2, folate receptor, IL-2 receptor, and VEGFR. In some embodiments, the targeting agent comprises an antibody that binds to a polypeptide selected from the group consisting of p53, Mucl, a mutated version of p53 that is present in breast cancer, HER-2, T and Tn haptens in glycoproteins of human breast carcinoma, and MSA breast carcinoma glycoprotein. In some embodiments, the targeting agent comprises an antibody selected from the group consisting of human carcinoma antigen, TP1 and TP3 antigens from osteocarcinoma cells, Thomsen-Friedenreich (TF) antigen from adenocarcinoma cells, KC-4 antigen from human prostrate adenocarcinoma, human colorectal cancer antigen, CA125 antigen from cystadenocarcinoma, DF3 antigen from human breast carcinoma, and p97 antigen of human melanoma, carcinoma or orosomucoid-related antigen. In some embodiments, the targeting agent is configured to permit the composition to cross the blood brain barrier. In some embodiments, the targeting agent is transferrin. In some embodiments, the targeting agent is configured to permit the composition to bind with a neuron within the central nervous system. In some embodiments, the targeting agent is a synthetic tetanus toxin fragment. In some embodiments, the synthetic tetanus toxin fragment comprises an amino acid peptide fragment. In some embodiments, the amino acid peptide fragment is HLNILSTLWKYR (SEQ ID NO: 2).

In some embodiments, the ligand comprises a trigger agent. The present invention is not limited to particular type or kind of trigger agent. In some embodiments, the trigger agent is configured to have a function such as, for example, a) a delayed release of a functional group from the dendrimer, b) a constitutive release of the therapeutic agent from the dendrimer, c) a release of a functional group from the dendrimer under conditions of acidosis, d) a release of a functional group from a dendrimer under conditions of hypoxia, and e) a release of the therapeutic agent from a dendrimer in the presence of a brain enzyme. Examples of trigger agents include, but are not limited to, an ester bond, an amide bond, an ether bond, an indoquinone, a nitroheterocyle, and a nitroimidazole.

In certain embodiments, the present invention provides improved methods for monitoring nanoparticles within biological settings. Nanoparticles have been used to monitor and modulate models of human disease and have great potential to transform clinical medicine. As nanoparticle platforms continue to evolve, understanding how these platforms interact with biological systems is critical. However, because of their unique properties, monitoring nanoparticles in complex biological environments can be difficult.

The present invention is not limited to particular types and/or kinds of imaging agents. Examples of imaging agents include, but are not limited to, fluorescein isothiocyanate (FITC), 6-TAMARA, acridine orange, and cis-parinaric acid. In certain embodiments, the present invention relates to alkyne-derivatized chemical reporters (e.g., imaging agents) that enable the rapid detection of azide-derivatized dendrimer nanoparticles in biological systems via copper catalyzed 1, 3 dipolar cycloaddition reaction. For example, by covalently coupling a fluorescent reporter to the nanoparticle scaffold after cellular delivery, this strategy allows tracking of the nanoparticles without having to synthesize distinct reporter functionalized nanoparticles. While not limited to particular uses, this strategy was used to monitor the behavior of dendrimer nanoparticles in diverse cellular environments. This strategy was shown to be able to monitor trafficking of dendrimer conjugates in a murine model of inflammation. Such experiments demonstrated the utility of small chemical reporters to monitor nanoparticle platforms following their delivery to intracellular targets in complex biological systems.

In certain embodiments, the present invention provides methods for synthesizing a functionalized dendrimer within a biological setting. The methods are not limited to particular steps. In some embodiments, the methods involve administering of one or more alkyne-derivitized dendrimers into a biological setting followed by administering of one or more azide-derivitized functional groups into the biological setting such that, upon contact, the functional groups conjugate with the dendrimer via copper catalyzed 1, 3 dipolar cycloaddition reactions. In some embodiments, the alkyne-derivitized dendrimers are further conjugated prior to administration into the biological setting with one or more functional groups. The methods are not limited to particular functional groups. The methods are not limited to particular biological settings.

The methods are not limited to a particular technique for monitoring. In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce when excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments, biosensor-comprising pro-drug complexes are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic biosensors for pH, oxygen concentration, Ca²+ concentration, and other physiologically relevant analytes.

In certain embodiments, the present invention provides methods for treating a disorder comprising administering to a subject one or more alkyne-derivatized dendrimers and one or more azide-derivatized functional groups. In some embodiments, the azide-derivatized functional group is a therapeutic agent known to be effective in treating the disorder. In some embodiments, the alkyne-derivatized dendrimers are conjugated with one or more functional groups prior to administration to the subject. In some embodiments, the alkyne-derivatized dendrimers are conjugated with therapeutic agents known to be effective in treating the disorder and the azide-derivatized functional group is a targeting agent. In some embodiments, the alkyne-derivatized dendrimers are conjugated with therapeutic agents known to be effective in treating the disorder and the azide-derivatized functional group is an imaging agent. In some embodiments, the alkyne-derivatized dendrimers are conjugated with imaging agents and the azide-derivatized functional group is a therapeutic agent known to be effective in treating the disorder. In some embodiments, the alkyne-derivatized dendrimers are conjugated with targeting agents and the azide-derivatized functional group is a therapeutic agent known to be effective in treating the disorder. The methods are not limited to particular disorders. In some embodiments, the disorder is selected from the group consisting of any type of cancer or cancer-related disorder (e.g., tumor, a neoplasm, a lymphoma, or a leukemia), a neoplastic disease, osteoarthritis, rheumatoid arthritis, septic arthritis, gout and pseudo-gout, juvenile idiopathic arthritis, psoriatic arthritis, Still's disease, and ankylosing spondylitis, comprising administering to a subject suffering from the disorder a dendrimer generated with the methods of the present invention. In some embodiments, the alkyne-derivatized dendrimer is co-administered with an additional agent(s) (e.g., therapeutic agents) so as to enhance such a treatment.

In certain embodiments, the present invention provides methods for monitoring a functionalized dendrimer within a biological setting. For example, in some embodiments, the methods involve administering alkyne-derivatized dendrimers to a biological setting (e.g., a cell sample, a human being) along with azide-derivatized imaging agents, wherein upon such administration, the imaging agents conjugate with the dendrimers via copper catalyzed 1, 3 dipolar cycloaddition reactions, thereby permitting the monitoring of the dendrimer via the imaging agent. In some embodiments, the alkyne-derivatized dendrimers are conjugated with one or more functional groups (e.g., therapeutic agents, targeting agents, etc.) prior to administration to the biological setting.

Additional embodiments will be apparent to persons skilled in the relevant art based on the teachings contained herein.

### DESCRIPTION OF THE DRAWINGS

FIG. 1 shows a schematic illustration of a bioorthogonal reporter embodiment for monitoring nanoparticles using a Cu(I) catalyzed azide-alkyne cycloaddition reaction.

FIG. 2 shows in vitro demonstration of receptor mediated endocytosis in cultured KB cells. (A) KB cells were incubated with increasing concentrations of FA-dendrimer and non-targeted dendrimer conjugates at 37° C. for 1 hour. The cells were harvested, fixed, and permeabilized prior to detection of the alkyne functionalized nanoparticles using Alexa647-azide. (B) Inhibition of folate dendrimer alkyne conjugate uptake by 30 minutes preincubation with 100 μM folic acid. Results are representative of two independent experiments.

FIG. 3 shows confocal microscopy demonstrating the flexibility of the bioorthogonal reporter system. (A) KB cells were incubated with 30 nM folate dendrimer alkyne conjugate and stained with DAPI and Alexa555-azide. (B) KB cells were incubated with the same dendrimer conjugate as (A). This treatment was then stained with DAPI and Alexa488-azide.

FIG. 4 shows bone marrow derived macrophages (BMDM) under diverse polarizing conditions. (A) The expression of macrophage mannose receptor (MMR) in BMDM treated with IL-4 and IL-10 was analyzed by qRT-PCR. (B) IL4 and IL10 differentiated BMDMs were incubated with 30 nM mannose-targeted and non-targeted dendrimer conjugates for 6 hours at 37° C. BMDMs were harvested and dendrimer conjugates were detected using AlexaFluor 647-azide reporter and analyzed by flow cytometry. Results are representative of two independent experiments. Of note, for the graph shown in FIG. 4B, the highest peak represents, “IL4”, the left-most peak represents, “Blank”, and the middle peak represents, “IL10.”

FIGS. 5A and 5B show in vitro characterization of methotrexate dendrimer therapeutic conjugates. FRα over-expressing KB cells were incubated with 30 nM of folate targeted methotrexate and methotrexate only dendrimer conjugates. Cells were harvested, fixed, and permeabilized at various time points. Therapeutic nanoparticles were detected in situ using Alexa647-azide. Results are representative of two independent experiments. Of note, for the first graph shown in FIG. 5A (the left-most graph), the highest peak is “Blank”, the second highest peak is “1 hour”, the third highest peak is “4 hours”, and the fourth highest peak is “24 hours.” For the second graph shown in FIG. 5A (the right-most graph), the shortest peak is “24 hours,” the left-most peak represents “Blank” and “4 hours”, and the middle peak represents, “1 hour.”

FIG. 6 shows in vivo monitoring of dendrimer conjugates using the bioorthogonal reporter system. Mice (n=2) were stimulated with thioglycollate IP. 3 days following thioglycollate, mice were administered 200 μl (35 mg/kg) folate functionalized dendrimer alkyne scaffolds. One day later (day 4), peritoneal macrophages (PEM), spleen, cervical lymph nodes, and mesenteric lymph nodes were isolated and processed into single-cell suspensions. Suspensions were stained with a CD11b monoclonal PE antibody and then fixed and permeabilized. Cell suspensions were then reacted with AlexaFluor 647-azide to detect dendrimer conjugates and analyzed by flow cytometry.

FIG. 7 shows a scheme for chemical synthesis of folate and mannose functionalized, dendrimer-alkyne conjugates.

FIG. 8 shows a schematic illustration of click efficiency tests using 3-azido-7-hydroxy coumarin fluorescent assay.

FIG. 9 presents a table describing click efficiency using 3-azido-7-hydroxy coumarin fluorescent assay.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “initial diagnosis” refers to a test result of initial cancer diagnosis that reveals the presence or absence of cancerous cells (e.g., using a biopsy and histology).

As used herein, the term “identifying the risk of said tumor metastasizing” refers to the relative risk (e.g., the percent chance or a relative score) of a tumor metastasizing.

As used herein, the term “identifying the risk of said tumor recurring” refers to the relative risk (e.g., the percent chance or a relative score) of a tumor recurring in the same organ as the original tumor.

As used herein, the term “subject at risk for cancer” refers to a subject with one or more risk factors for developing a specific cancer. Risk factors include, but are not limited to, gender, age, genetic predisposition, environmental expose, and previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue and the stage of the cancer.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer on a subject's future health (e.g., expected morbidity or mortality, the likelihood of getting cancer, and the risk of metastasis).

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “sample” is used in its broadest sense. In one sense, it is meant to include a specimen or culture obtained from any source, as well as biological and environmental samples. Biological settings generally pertain to biological samples (e.g., in vitro, in vivo, in situ, ex vivo). Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “drug” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “purified” or “to purify” or “compositional purity” refers to the removal of components (e.g., contaminants) from a sample or the level of components (e.g., contaminants) within a sample. For example, unreacted moieties, degradation products, excess reactants, or byproducts are removed from a sample following a synthesis reaction or preparative method.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using screening methods known in the art.

As used herein, the term “nanodevice” or “nanodevices” refer, generally, to compositions comprising dendrimers of the present invention. As such, a nanodevice may refer to a composition comprising a dendrimer of the present invention that may contain one or more ligands, linkers, and/or functional groups (e.g., a therapeutic agent, a targeting agent, a trigger agent, an imaging agent) conjugated to the dendrimer.

As used herein, the term “degradable linkage,” when used in reference to a polymer refers to a conjugate that comprises a physiologically cleavable linkage (e.g., a linkage that can be hydrolyzed (e.g., in vivo) or otherwise reversed (e.g., via enzymatic cleavage). Such physiologically cleavable linkages include, but are not limited to, ester, carbonate ester, carbamate, sulfate, phosphate, acyloxyalkyl ether, acetal, and ketal linkages (See, e.g., U.S. Pat. No. 6,838,076, herein incorporated by reference in its entirety). Similarly, the conjugate may comprise a cleavable linkage present in the linkage between the dendrimer and functional group, or, may comprise a cleavable linkage present in the polymer itself (See, e.g., U.S. Pat. App. Nos. 20050158273 and 20050181449, each of which is herein incorporated by reference in its entirety).

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond (e.g., typically a covalent bond) that is substantially stable in water (i.e., does not undergo hydrolysis under physiological conditions to any appreciable extent over an extended period of time). Examples of hydrolytically stable linkages include, but are not limited to, carbon-carbon bonds (e.g., in aliphatic chains), ethers, amides, urethanes, and the like.

As used herein, the term “NAALADase inhibitor” refers to any one of a multitude of inhibitors for the neuropeptidase NAALADase (N-acetylated-alpha linked acidic dipeptidase). Such inhibitors of NAALADase have been well characterized. For example, an inhibitor can be selected from the group comprising, but not limited to, those found in U.S. Pat. No. 6,011,021, herein incorporated by reference in its entirety.

As used herein, the term “Baker-Huang dendrimer” or “Baker-Huang PAMAM dendrimer” refers to a dendrimer comprised of branching units of structure:

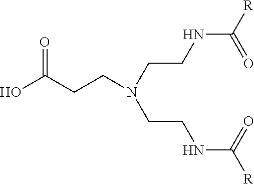

wherein R comprises a carbon-containing functional group (e.g., CF₃). In some embodiments, the branching unit is activated to its HNS ester. In some embodiments, such activation is achieved using TSTU. In some embodiments, EDA is added. In some embodiments, the dendrimer is further treated to replace, e.g., CF₃ functional groups with NH₂ functional groups; for example, in some embodiments, a CF₃-containing version of the dendrimer is treated with K₂CO₃ to yield a dendrimer with terminal NH₂ groups (for example, as shown in Scheme 2). In some embodiments, terminal groups of a Baker-Huang dendrimer are further derivatized and/or further conjugated with other moieties. For example, one or more functional ligands (e.g., for therapeutic, targeting, imaging, or drug delivery function(s)) may be conjugated to a Baker-Huang dendrimer, either via direct conjugation to terminal branches or indirectly (e.g., through linkers, through other functional groups (e.g., through an OH— functional group)). In some embodiments, the order of iterative repeats from core to surface is amide bonds first, followed by tertiary amines, with ethylene groups intervening between the amide bond and tertiary amines. In preferred embodiments, a Baker-Huang dendrimer is synthesized by convergent synthesis methods.

As used herein, the term “click chemistry” refers to chemistry tailored to generate substances quickly and reliably by joining small modular units together (see, e.g., Kolb et al. (2001) Angewandte Chemie Intl. Ed. 40:2004-2011; Evans (2007) Australian J. Chem. 60:384-395; Carlmark et al. (2009) Chem. Soc. Rev. 38:352-362; each herein incorporated by reference in its entirety).

As used herein, the term “scaffold” refers to a compound to which other moieties are attached (e.g., conjugated). In some embodiments, a scaffold is conjugated to bioactive functional conjugates (e.g., a therapeutic agent, a targeting agent, a trigger agent, an imaging agent). In some embodiments, a scaffold is conjugated to a dendrimer (e.g., a PAMAM dendrimer). In some embodiments, conjugation of a scaffold to a dendrimer and/or a functional conjugate(s) is direct, while in other embodiments conjugation of a scaffold to a dendrimer and/or a functional conjugate(s) is indirect, e.g., an intervening linker is present between the scaffold compound and the dendrimer, and/or the scaffold and the functional conjugate(s).

As used herein, the term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, conjugation between a dendrimer (e.g., a terminal arm of a dendrimer) and a functional ligand is accomplished during a “one-pot” reaction. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino) pyridine) (see, e.g., International Patent Application No. PCT/US2010/042556, herein incorporated by reference in its entirety).

As used herein, the term “solvent” refers to a medium in which a reaction is conducted. Solvents may be liquid but are not limited to liquid form. Solvent categories include but are not limited to nonpolar, polar, protic, and aprotic.

As used herein, the term “dialysis” refers to a purification method in which the solution surrounding a substance is exchanged over time with another solution. Dialysis is generally performed in liquid phase by placing a sample in a chamber, tubing, or other device with a selectively permeable membrane. In some embodiments, the selectively permeable membrane is cellulose membrane. In some embodiments, dialysis is performed for the purpose of buffer exchange. In some embodiments, dialysis may achieve concentration of the original sample volume. In some embodiments, dialysis may achieve dilution of the original sample volume.

As used herein, the term “precipitation” refers to purification of a substance by causing it to take solid form, usually within a liquid context. Precipitation may then allow collection of the purified substance by physical handling, e.g. centrifugation or filtration.

As used herein, an “ester coupling agent” refers to a reagent that can facilitate the formation of an ester bond between two reactants. The present invention is not limited to any particular coupling agent or agents. Examples of coupling agents include but are not limited to 2-chloro-1-methylpyridium iodide and 4-(dimethylamino) pyridine, or dicyclohexylcarbodiimide and 4-(dimethylamino) pyridine or diethyl azodicarboxylate and triphenylphosphine or other carbodiimide coupling agent and 4-(dimethylamino)pyridine.

As used herein, the term “glycidolate” refers to the addition of a 2,3-dihydroxylpropyl group to a reagent using glycidol as a reactant. In some embodiments, the reagent to which the 2,3-dihydroxylpropyl groups are added is a dendrimer. In some embodiments, the dendrimer is a PAMAM dendrimer. Glycidolation may be used generally to add terminal hydroxyl functional groups to a reagent.

As used herein, the term “ligand” or “functional group” refers to any moiety covalently attached (e.g., conjugated) to a dendrimer branch; in preferred embodiments, such conjugation is indirect (e.g., an intervening moiety exists between the dendrimer branch and the ligand) rather than direct (e.g., no intervening moiety exists between the dendrimer branch and the ligand). Indirect attachment of a ligand to a dendrimer may exist where a scaffold compound (e.g., triazine scaffold) intervenes. In preferred embodiments, ligands have functional utility for specific applications, e.g., for therapeutic, targeting, imaging, or drug delivery function(s). The terms “ligand”, “conjugate”, and “functional group” may be used interchangeably.

### DETAILED DESCRIPTION OF THE INVENTION

Since the introduction of “click” chemistry, these bioorthogonal reactions have had a significant impact on organic synthesis and macromolecular chemistry (see, e.g., Kolb, H. C., et al., Angew Chem Int Ed Engl 40, 2004-2021 (2001); Wang, Q. et al. J Am Chem Soc 125, 3192-3193, doi:10.1021/ja021381e (2003); each herein incorporated by reference in their entireties). The efficiency, specificity, and modularity afforded by these reporters have led to significant advancements in both small molecule synthesis and polymer chemistry. The Cu(I)-catalyzed azide-alkyne cycloaddition (CuAAC) “click” reactions have been used as a chemical reporters to monitor DNA synthesis, metabolite flux, and for proteomics applications (see, e.g., Baskin, J. M. et al. Proc Natl Acad Sci USA 104, 16793-16797 (2007); Chang, P. V. et al. Angew Chem Int Ed Engl 48, 4030-4033 (2009); Prescher, J. A. & Bertozzi, C. R. Nat Chem Biol 1, 13-21; each herein incorporated by reference in their entireties). For example, a click chemistry based reporter has been used to monitor DNA synthesis in culture and animal models (see, e.g., Salic, A. & Mitchison, T. J., Proc Natl Acad Sci USA 105, 2415-2420; herein incorporated by reference in its entirety). More recently, a copper-free click chemistry reporter strategy was used to monitor cell surface modifications in mice further highlighting to potential of bioorthogonal reporters to monitor biological processes both in vitro and in vivo (see, e.g., Chang, P. V., et al., J Am Chem Soc 129, 8400-8401 (2007); Baskin, J. M. et al. Proc Natl Acad Sci USA 104, 16793-16797; each herein incorporated by reference in their entireties). In the diagnostics field, a catalyst-free cycloaddition reaction in combination with antibody recognition to detect cancer cells was used (see, e.g., Haun, J. B., et al., Nat Nanotechnol 5, 660-665; herein incorporated by reference in its entirety). The efficiency and the mild conditions involved in these reactions coupled with the small size of the chemical reporters are significant advantages for these types of monitoring systems.

In certain embodiments, the present invention relates to compositions (e.g., dendrimer scaffolds) capable of click chemistry for use in synthesis of functionalized dendrimers within biological settings, and methods of use of the same. In particular, the present invention relates to conjugating alkyne-derivatized functional groups (e.g., ligands) via copper catalyzed 1, 3 dipolar cycloaddition reaction with azide-derivatized dendrimer nanoparticles within biological settings (e.g., administering an alkyne-derivatized functional group to a biological setting already having an azide-derivatized dendrimer such that, via click chemistry, the functional group conjugates with the dendrimer within the biological setting).

Indeed, embodiments of the present invention leverage the properties of the CuAAC reaction to monitor dendrimer nanoparticles in biological systems. For example, during the synthesis of dendrimer conjugates of the present invention, alkyne linkers were included on the dendrimer scaffold to allow for downstream detection following their introduction into biological systems. These linkers allowed monitor of nanoparticles following their delivery to cellular systems and in vivo models. After the cellular delivery of the dendrimer conjugates, the CuAAC reaction was utilized to couple a fluorescent azide probe to the alkyne functionalized dendrimer scaffolds to assemble the fluorescent reporter. This bioorthogonal reporter system also provided the flexibility to change fluorescent labels without synthesizing new reporter functionalized dendrimer conjugates. More generally, this strategy can be used to efficiently monitor nanoparticle platforms in complex biological environments without perturbing intrinsic properties and without resource intensive synthetic strategies.

The present invention is not limited to the use of particular types and/or kinds of dendrimers (e.g., a dendrimer conjugated with at least one functional group). Indeed, dendrimeric polymers have been described extensively (See, e.g., Tomalia, Advanced Materials 6:529 (1994); Angew, Chem. Int. Ed. Engl., 29:138 (1990); incorporated herein by reference in their entireties). Dendrimer polymers are synthesized as defined spherical structures typically ranging from 1 to 20 nanometers in diameter. Methods for manufacturing a G5 PAMAM dendrimer with a protected core are known (U.S. patent application Ser. No. 12/403,179; herein incorporated by reference in its entirety). In preferred embodiments, the protected core diamine is NH₂—CH₂—CH₂—NHPG. Molecular weight and the number of terminal groups increase exponentially as a function of generation (the number of layers) of the polymer. In some embodiments of the present invention, half generation PAMAM dendrimers are used. For example, when an ethylenediamine (EDA) core is used for dendrimer synthesis, alkylation of this core through Michael addition results in a half-generation molecule with ester terminal groups; amidation of such ester groups with excess EDA results in creation of a full-generation, amine-terminated dendrimer (Majoros et al., Eds. (2008) Dendrimer-based Nanomedicine, Pan Stanford Publishing Pte. Ltd., Singapore, p. 42). Different types of dendrimers can be synthesized based on the core structure that initiates the polymerization process. In some embodiments, the PAMAM dendrimers are “Baker-Huang dendrimers” or “Baker-Huang PAMAM dendrimers” (see, e.g., U.S. Provisional Patent Application Ser. No. 61/251,244; herein incorporated by reference in its entirety).

The dendrimer core structures dictate several characteristics of the molecule such as the overall shape, density and surface functionality (See, e.g., Tomalia et al., Chem. Int. Ed. Engl., 29:5305 (1990)). Spherical dendrimers can have ammonia as a trivalent initiator core or ethylenediamine (EDA) as a tetravalent initiator core. Recently described rod-shaped dendrimers (See, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for GMP applications and in vivo usage.

Numerous U.S. Patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describes methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

PAMAM dendrimers are highly branched, narrowly dispersed synthetic macromolecules with well-defined chemical structures. PAMAM dendrimers can be easily modified and conjugated with multiple functionalities such as targeting molecules, imaging agents, and drugs (Thomas et al. (2007) Poly(amidoamine) Dendrimer-based Multifunctional Nanoparticles, in Nanobiotechnology: Concepts, Methods and Perspectives, Merkin, Ed., Wiley-VCH; herein incorporated by reference in its entirety). They are water soluble, biocompatible, and cleared from the blood through the kidneys (Peer et al. (2007) Nat. Nanotechnol. 2:751-760; herein incorporated by reference in its entirety) which eliminates the need for biodegradability. Because of these desirable properties, PAMAM dendrimers have been widely investigated for drug delivery (Esfand et al. (2001) Drug Discov. Today 6:427-436; Patri et al. (2002) Curr. Opin. Chem. Biol. 6:466-471; Kukowska-Latallo et al. (2005) Cancer Res. 65:5317-5324; Quintana et al. (2002) Pharmaceutical Res. 19:1310-1316; Thomas et al. (2005) J. Med. Chem. 48:3729-3735; each herein incorporated by reference in its entirety), gene therapy (Kukowska Latallo et al. (1996) PNAS 93:4897-4902; Eichman et al. (2000) Pharm. Sci. Technolo. Today 3:232-245; Luo et al. (2002) Macromol. 35:3456-3462; each herein incorporated by reference in its entirety), and imaging applications (Kobayashi et al. (2003) Bioconj. Chem. 14:388-394; herein incorporated by reference in its entirety).

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polyeation non-covalently coupled to the polynucleotide.

Dendrimer-antibody conjugates for use in in vitro diagnostic applications have previously been demonstrated (See, e.g., Singh et al., Clin. Chem., 40:1845 (1994)), for the production of dendrimer-chelant-antibody constructs, and for the development of boronated dendrimer-antibody conjugates (for neutron capture therapy); each of these latter compounds may be used as a cancer therapeutic (See, e.g., Wu et al., Bioorg. Med. Chem. Left., 4:449 (1994); Wiener et al., Magn. Reson. Med. 31:1 (1994); Barth et al., Bioconjugate Chem. 5:58 (1994); and Barth et al.).

Some of these conjugates have also been employed in the magnetic resonance imaging of tumors (See, e.g., Wu et al., (1994) and Wiener et al., (1994), supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (See, e.g., Duncan and Malik, Control Rel. Bioact. Mater. 23:105 (1996)).

Dendrimers have also been conjugated to fluorochromes or molecular beacons and shown to enter cells. They can then be detected within the cell in a manner compatible with sensing apparatus for evaluation of physiologic changes within cells (See, e.g., Baker et al., Anal. Chem. 69:990 (1997)). Finally, dendrimers have been constructed as differentiated block copolymers where the outer portions of the molecule may be digested with either enzyme or light-induced catalysis (See, e.g., Urdea and Hom, Science 261:534 (1993)). This allows the controlled degradation of the polymer to release therapeutics at the disease site and provides a mechanism for an external trigger to release the therapeutic agents.

In experiments conducted during the course of developing embodiments for the present invention, alkyne-derivatized functional groups (e.g., ligands) were conjugated azide-derivatized dendrimer nanoparticles via copper catalyzed 1, 3 dipolar cycloaddition reactions within biological settings (e.g., administering an alkyne-derivatized functional group to a biological setting already having an azide-derivatized dendrimer such that, via click chemistry, the functional group conjugates with the dendrimer within the biological setting).

In some embodiments, the azide-derivatized dendrimer has no functional groups. In some embodiments, the azide-derivatized dendrimer has thereon one or more functional groups. In some embodiments, such functional group(s) are attached with the dendrimer via a linker. The present invention is not limited to a particular type or kind of linker. In some embodiments, the linker comprises a spacer comprising between 1 and 8 straight or branched carbon chains. In some embodiments, the straight or branched carbon chains are unsubstituted. In some embodiments, the straight or branched carbon chains are substituted with alkyls.

For example, in some embodiments, following introduction of azide-derivatized dendrimers conjugated with therapeutic agents and/or targeting agents into a biological setting (e.g., a cell sample), alkyne-derivatized imaging agents are introduced for the purpose of monitoring/tracking the location of the dendrimer within the biological setting. Such techniques permit, for example, assimilation of the dendrimer (e.g., functionalized or non-functionalized) into a biological setting prior to conjugation of the functional group.

The present invention is not limited to particular azide moieties (or equivalents thereof). In some embodiments, the azide moiety comprises the formula N₃⁻.

The present invention is not limited to particular alkyne moieties (or equivalents thereof). In some embodiments, the alkyne moiety is a cyclooctyne moiety (or equivalents thereof). In some embodiments, the cyclooctyne moiety comprises the following formula:

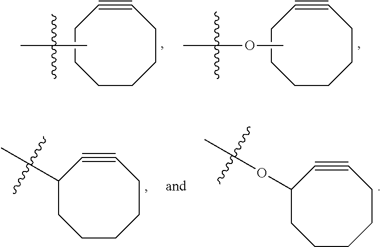

The present invention is not limited to a particular manner of conjugating the azide moieties (or equivalents thereof) and/or the alkyne moieties (or equivalents thereof) with either a dendrimer structure and/or a functional group. In some embodiments, the azide moieties (or equivalents thereof) and/or the alkyne moieties (or equivalents thereof) are conjugated with either a dendrimer and/or a functional group via a primary amine end group, a hydroxyl end group, a carboxylic acid end group, a thiol end group, etc.).

The present invention is not limited to a particular type and/or kind of biological setting for conjugating alkyne-derivatized functional groups with azide-derivatized dendrimer nanoparticles via copper catalyzed 1, 3 dipolar cycloaddition reactions. In some embodiments, the biological setting is either in vitro, in situ, or in vivo). Biological samples may be obtained from animals (including humans) and encompass fluids, solids, tissues, and gases. Biological samples include blood products, such as plasma, serum and the like.

The present invention is not limited to particular ligand types (e.g., functional groups). Examples of ligand types include but are not limited to therapeutic agents, targeting agents, trigger agents, and imaging agents. In some embodiments, the ligand is an azide ligand that includes an azido group. In some embodiments, the ligand includes an aromatic group. Methods, systems, and compositions of the present invention are not limited by the number of different ligand types used. There may be 1, 2, 3, 4, 5, 6, 7, 8, 9, 10 or more different types of ligands attached to a dendrimer molecule.

The present invention is not limited to the use of particular therapeutic agents. In some embodiments, the therapeutic agents are effective in treating autoimmune disorders and/or inflammatory disorders (e.g., arthritis). Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, parenteral gold or oral gold.

In some embodiments, the therapeutic agents are effective in treating cancer (see, e.g., U.S. Pat. Nos. 6,471,968, 7,078,461, and U.S. patent application Ser. Nos. 09/940,243, 10/431,682, 11/503,742, 11/661,465, 11/523,509, 12/403,179, 12/106,876, and 11/827,637; and U.S. Provisional Patent Application Ser. Nos. 61/256,759, 61/140,840, 61/091,608, 61/097,780, 61/101,461, 61/237,172, 61/229,168, 61/221,596, and 61/251,244; each herein incorporated by reference in their entireties).

In some embodiments, the ligand (e.g., therapeutic agent) is conjugated with the dendrimer and/or triazine compound via a trigger agent. The present invention is not limited to particular types or kinds of trigger agents.

In some embodiments, sustained release (e.g., slow release over a period of 24-48 hours) of the ligand (e.g., therapeutic agent) is accomplished through conjugating the therapeutic agent (e.g., directly) (e.g., indirectly through one or more additional functional groups) to a trigger agent that slowly degrades in a biological system (e.g., amide linkage, ester linkage, ether linkage). In some embodiments, constitutively active release of a therapeutic agent is accomplished through conjugating the therapeutic agent to a trigger agent that renders the therapeutic agent constitutively active in a biological system (e.g., amide linkage, ether linkage).

In some embodiments, release of a therapeutic agent under specific conditions is accomplished through conjugating the therapeutic agent (e.g., directly) (e.g., indirectly through one or more additional functional groups) to a trigger agent that degrades under such specific conditions (e.g., through activation of a trigger molecule under specific conditions that leads to release of the therapeutic agent). For example, once a conjugate (e.g., a therapeutic agent conjugated with a trigger agent and a targeting agent) arrives at a target site in a subject (e.g., a tumor, or a site of inflammation), components in the target site (e.g., a tumor associated factor, or an inflammatory or pain associated factor) interact with the trigger agent thereby initiating cleavage of the therapeutic agent from the trigger agent. In some embodiments, the trigger agent is configured to degrade (e.g., release the therapeutic agent) upon exposure to a tumor-associated factor (e.g., hypoxia and pH, an enzyme (e.g., glucuronidase and/or plasmin), a cathepsin, a matrix metalloproteinase, a hormone receptor (e.g., integrin receptor, hyaluronic acid receptor, luteinizing hormone-releasing hormone receptor, etc.), cancer and/or tumor specific DNA sequence), an inflammatory associated factor (e.g., chemokine, cytokine, etc.) or other moiety.

In some embodiments, the present invention provides a therapeutic agent conjugated with a trigger agent that is sensitive to (e.g., is cleaved by) hypoxia (e.g., indolequinone). Hypoxia is a feature of several disease states, including cancer, inflammation and rheumatoid arthritis, as well as an indicator of respiratory depression (e.g., resulting from analgesic drugs).

Advances in the chemistry of bioreductive drug activation have led to the design of various hypoxia-selective drug delivery systems in which the pharmacophores of drugs are masked by reductively cleaved groups. In some embodiments, the trigger agent is utilizes a quinone, N-oxide and/or (hetero)aromatic nitro groups. For example, a quinone present in a conjugate is reduced to phenol under hypoxia conditions, with spontaneous formation of lactone that serves as a driving force for drug release. In some embodiments, a heteroaromatic nitro compound present in a conjugate (e.g., a therapeutic agent conjugated (e.g., directly or indirectly) with a trigger agent) is reduced to either an amine or a hydroxylamine, thereby triggering the spontaneous release of a therapeutic agent. In some embodiments, the trigger agent degrades upon detection of reduced pO2 concentrations (e.g., through use of a redox linker).

The concept of pro-drug systems in which the pharmacophores of drugs are masked by reductively cleavable groups has been widely explored by many research groups and pharmaceutical companies (see, e.g., Beall, H. D., et al., Journal of Medicinal Chemistry, 1998. 41(24): p. 4755-4766; Ferrer, S., D. P. Naughton, and M. D. Threadgill, Tetrahedron, 2003. 59(19): p. 3445-3454; Naylor, M. A., et al., Journal of Medicinal Chemistry, 1997. 40(15): p. 2335-2346; Phillips, R. M., et al., Journal of Medicinal Chemistry, 1999. 42(20): p. 4071-4080; Zhang, Z., et al., Organic & Biomolecular Chemistry, 2005. 3(10): p. 1905-1910; each of which are herein incorporated by reference in their entireties). Several such hypoxia activated pro-drugs have been advanced to clinical investigations, and work in relevant oxygen concentrations to prevent cerebral damage. The present invention is not limited to particular hypoxia activated trigger agents. In some embodiments, the hypoxia activated trigger agents include, but are not limited to, indolequinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770; each herein incorporated by reference in their entireties).

In some embodiments, the trigger agent is sensitive to (e.g., is cleaved by) and/or associates with a tumor-associated enzyme. For example, in some embodiments, the trigger agent that is sensitive to (e.g., is cleaved by) and/or associates with a glucuronidase. Glucuronic acid can be attached to several anticancer drugs via various linkers. These anticancer drugs include, but are not limited to, doxorubicin, paclitaxel, docetaxel, 5-fluorouracil, 9-aminocamtothecin, as well as other drugs under development. These pro-drugs are generally stable at physiological pH and are significantly less toxic than the parent drugs.

In some embodiments, the trigger agent is sensitive to (e.g., is cleaved by) and/or associates with brain enzymes. For example, trigger agents such as indolequinone are reduced by brain enzymes such as, for example, diaphorase (DT-diaphorase) (see, e.g., Damen, E. W. P., et al., Bioorganic & Medicinal Chemistry, 2002. 10(1): p. 71-77; herein incorporated by reference in its entirety). For example, in such embodiments, the antagonist is only active when released during hypoxia to prevent respiratory failure.

In some embodiments, the trigger agent is sensitive to (e.g., is cleaved by) and/or associates with a protease. The present invention is not limited to any particular protease. In some embodiments, the protease is a cathepsin. In some embodiments, a trigger comprises a Lys-Phe-PABC moiety (e.g., that acts as a trigger). In some embodiments, a Lys-Phe-PABC moiety linked to doxorubicin, mitomycin C, and paclitaxel are utilized as a trigger-therapeutic conjugate in a dendrimer conjugate provided herein (e.g., that serve as substrates for lysosomal cathepsin B or other proteases expressed (e.g., overexpressed) in tumor cells. In some embodiments, utilization of a 1,6-elimination spacer/linker is utilized (e.g., to permit release of therapeutic drug post activation of trigger).

In some embodiments, the trigger agent is sensitive to (e.g., is cleaved by) and/or associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the trigger agent is sensitive to (e.g., is cleaved by) and/or associates with a matrix metalloprotease (MMP). In some embodiments, the trigger agent is sensitive to (e.g., is cleaved by) and/or that associates with β-Lactamase (e.g., a β-Lactamase activated cephalosporin-based pro-drug).

In some embodiments, the trigger agent is sensitive to (e.g., is cleaved by) and/or activated by a receptor (e.g., expressed on a target cell (e.g., a tumor cell)).

In some embodiments, the trigger agent is sensitive to (e.g., is cleaved by) and/or activated by a nucleic acid. Nucleic acid triggered catalytic drug release can be utilized in the design of chemotherapeutic agents. Thus, in some embodiments, disease specific nucleic acid sequence is utilized as a drug releasing enzyme-like catalyst (e.g., via complex formation with a complimentary catalyst-bearing nucleic acid and/or analog). In some embodiments, the release of a therapeutic agent is facilitated by the therapeutic component being attached to a labile protecting group, such as, for example, cisplatin or methotrexate being attached to a photolabile protecting group that becomes released by laser light directed at cells emitting a color of fluorescence (e.g., in addition to and/or in place of target activated activation of a trigger component of a dendrimer conjugate). In some embodiments, the therapeutic device also may have a component to monitor the response of the tumor to therapy. For example, where a therapeutic agent of the dendrimer induces apoptosis of a target cell (e.g., a cancer cell (e.g., a prostate cancer cell)), the caspase activity of the cells may be used to activate a green fluorescence. This allows apoptotic cells to turn orange, (combination of red and green) while residual cells remain red. Any normal cells that are induced to undergo apoptosis in collateral damage fluoresce green.

In some embodiments, a dendrimer is conjugated (e.g., directly or indirectly (e.g., via a triazine compound)) with a targeting agent. The present invention is not limited to any particular targeting agent. In some embodiments, targeting agents are conjugated to a dendrimer (eg.., directly or indirectly) for delivery to desired body regions (e.g., to the central nervous system (CNS); to a tumor). The targeting agents are not limited to targeting specific body regions.

In some embodiments, the targeting agent is a moiety that has affinity for a tumor associated factor. For example, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)).

The present invention is not limited to cancer and/or tumor targeting agents. Indeed, multifunctional dendrimers can be targeted (e.g., via a linker conjugated to the dendrimer wherein the linker comprises a targeting agent) to a variety of target cells or tissues (e.g., to a biologically relevant environment) via conjugation to an appropriate targeting agent. For example, in some embodiments, the targeting agent is a moiety that has affinity for an inflammatory factor (e.g., a cytokine or a cytokine receptor moiety (e.g., TNF-α receptor)). In some embodiments, the targeting agent is a sugar, peptide, antibody or antibody fragment, hormone, hormone receptor, or the like.

In some embodiments of the present invention, the targeting agent includes, but is not limited to an antibody, receptor ligand, hormone, vitamin, and antigen, however, the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

Antibodies can be generated to allow for the targeting of antigens or immunogens (e.g., tumor, tissue or pathogen specific antigens) on various biological targets (e.g., pathogens, tumor cells, normal tissue). Such antibodies include, but are not limited to polyclonal, monoclonal, chimeric, single chain, Fab fragments, and an Fab expression library.

In some embodiments, the targeting agent is an antibody. In some embodiments, the antibodies recognize, for example, tumor-specific epitopes (e.g., TAG-72 (See, e.g., Kjeldsen et al., Cancer Res. 48:2214-2220 (1988); U.S. Pat. Nos. 5,892,020; 5,892,019; and 5,512,443; each herein incorporated by reference in their entireties); human carcinoma antigen (See, e.g., U.S. Pat. Nos. 5,693,763; 5,545,530; and 5,808,005; each herein incorporated by reference in their entireties); TP1 and TP3 antigens from osteocarcinoma cells (See, e.g., U.S. Pat. No. 5,855,866; herein incorporated by reference in its entirety); Thomsen-Friedenreich (TF) antigen from adenocarcinoma cells (See, e.g., U.S. Pat. No. 5,110,911; herein incorporated by reference in its entirety); “KC-4 antigen” from human prostrate adenocarcinoma (See, e.g., U.S. Pat. Nos. 4,708,930 and 4,743,543; each herein incorporated by reference in their entireties); a human colorectal cancer antigen (See, e.g., U.S. Pat. No. 4,921,789; herein incorporated by reference in its entirety); CA125 antigen from cystadenocarcinoma (See, e.g., U.S. Pat. No. 4,921,790; herein incorporated by reference in its entirety); DF3 antigen from human breast carcinoma (See, e.g., U.S. Pat. Nos. 4,963,484 and 5,053,489; each herein incorporated by reference in their entireties); a human breast tumor antigen (See, e.g., U.S. Pat. No. 4,939,240: herein incorporated by reference in its entirety); p97 antigen of human melanoma (See, e.g., U.S. Pat. No. 4,918,164: herein incorporated by reference in its entirety); carcinoma or orosomucoid-related antigen (CORA)(See, e.g., U.S. Pat. No. 4,914,021; herein incorporated by reference in its entirety); a human pulmonary carcinoma antigen that reacts with human squamous cell lung carcinoma but not with human small cell lung carcinoma (See, e.g., U.S. Pat. No. 4,892,935; herein incorporated by reference in its entirety); T and Tn haptens in glycoproteins of human breast carcinoma (See, e.g., Springer et al., Carbohydr. Res. 178:271-292 (1988); herein incorporated by reference in its entirety), MSA breast carcinoma glycoprotein termed (See, e.g., Tjandra et al., Br. J. Surg. 75:811-817 (1988); herein incorporated by reference in its entirety); MFGM breast carcinoma antigen (See, e.g., Ishida et al., Tumor Biol. 10:12-22 (1989); herein incorporated by reference in its entirety); DU-PAN-2 pancreatic carcinoma antigen (See, e.g., Lan et al., Cancer Res. 45:305-310 (1985); herein incorporated by reference in its entirety); CA125 ovarian carcinoma antigen (See, e.g., Hanisch et al., Carbohydr. Res. 178:29-47 (1988); herein incorporated by reference in its entirety); YH206 lung carcinoma antigen (See, e.g., Hinoda et al., (1988) Cancer J. 42:653-658 (1988); herein incorporated by reference in its entirety).

In some embodiments, the targeting agents target the central nervous system (CNS). In some embodiments, where the targeting agent is specific for the CNS, the targeting agent is transferrin (see, e.g., Daniels, T. R., et al., Clinical Immunology, 2006. 121(2): p. 159-176; Daniels, T. R., et al., Clinical Immunology, 2006. 121(2): p. 144-158; each herein incorporated by reference in their entireties). Transferrin has been utilized as a targeting vector to transport, for example, drugs, liposomes and proteins across the blood-brain barrier (BBB) by receptor mediated transcytosis (see, e.g., Smith, M. W. and M. Gumbleton, Journal of Drug Targeting, 2006. 14(4): p. 191-214; herein incorporated by reference in its entirety). In some embodiments, the targeting agents target neurons within the central nervous system (CNS). In some embodiments, where the targeting agent is specific for neurons within the CNS, the targeting agent is a synthetic tetanus toxin fragment (e.g., a 12 amino acid peptide (Tet 1) (HLNILSTLWKYR) (SEQ ID NO: 2)) (see, e.g., Liu, J. K., et al., Neurobiology of Disease, 2005. 19(3): p. 407-418; herein incorporated by reference in its entirety).

In certain embodiments, the present invention provides improved methods for monitoring nanoparticles within biological settings. A multiplicity of imaging agents find use in the present invention. Examples of imaging agents include, but are not limited to, fluorescein isothiocyanate (FITC), 6-TAMARA, acridine orange, and cis-parinaric acid. In certain embodiments, the present invention relates to alkyne-derivatized chemical reporters (e.g., imaging agents) that enable the rapid detection of azide-derivatized dendrimer nanoparticles in biological systems via copper catalyzed 1, 3 dipolar cycloaddition reaction. For example, by covalently coupling a fluorescent reporter to the nanoparticle scaffold after cellular delivery, this strategy allows tracking of the nanoparticles without having to synthesize distinct reporter functionalized nanoparticles. While not limited to particular uses, this strategy was used to monitor the behavior of dendrimer nanoparticles in diverse cellular environments. This strategy was shown to be able to monitor trafficking of dendrimer conjugates in a murine model of inflammation. Such experiments demonstrated the utility of small chemical reporters to monitor nanoparticle platforms following their delivery to intracellular targets in complex biological systems. In some embodiments of the present invention, imaging modules comprise surface modifications of quantum dots (See e.g., Chan and Nie, Science 281:2016 (1998)) such as zinc sulfide-capped cadmium selenide coupled to biomolecules (Sooklal, Adv. Mater., 10:1083 (1998)).

In some embodiments, once a component(s) of a targeted multifunctional dendrimer has attached to (or been internalized into) a target cell (e.g., tumor cell and or inflammatory cell), one or more modules on serves to image its location. In some embodiments, chelated paramagnetic ions, such as Gd(III)-diethylenetriaminepentaacetic acid (Gd(III)-DTPA), are conjugated to the multifunctional dendrimer. Other paramagnetic ions that may be useful in this context include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof.

Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

Multifunctional dendrimers allow functional microscopic imaging of tumors and provide improved methods for imaging. The methods find use in vivo, in vitro, and ex vivo. For example, in one embodiment, dendrimer functional groups are designed to emit light or other detectable signals upon exposure to light. Although the labeled functional groups may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (See, e.g., Farkas et al., SPEI 2678:200 (1997); herein incorporated by reference in its entirety). In embodiments where the imaging agents are present in deeper tissue, longer wavelengths in the Near-infrared (NMR) are used (See e.g., Lester et al., Cell Mol. Biol. 44:29 (1998); herein incorporated by reference in its entirety). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce when excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments, biosensor-comprising pro-drug complexes are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic biosensors for pH, oxygen concentration, Ca²+ concentration, and other physiologically relevant analytes.

In these embodiments, fluorescent groups such as fluorescein are employed in the imaging agent. Fluorescein is easily attached to the dendrimer surface via the isothiocyanate derivatives, available from MOLECULAR PROBES, Inc. This allows the multifunctional dendrimer or components thereof to be imaged with the cells via confocal microscopy. Sensing of the effectiveness of the multifunctional dendrimer or components thereof is preferably achieved by using fluorogenic peptide enzyme substrates. For example, apoptosis caused by the therapeutic agent results in the production of the peptidase caspase-1 (ICE). CALBIOCHEM sells a number of peptide substrates for this enzyme that release a fluorescent moiety. A particularly useful peptide for use in the present invention is: MCA-Tyr-Glu-Val-Asp-Gly-Trp-Lys-(DNP)-NH₂ (SEQ ID NO: 1) where MCA is the (7-methoxycoumarin-4-yl)acetyl and DNP is the 2,4-dinitrophenyl group (See, e.g., Talanian et al., J. Biol. Chem., 272: 9677 (1997); herein incorporated by reference in its entirety). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm). In some embodiments, the lysine end of the peptide is linked to pro-drug complex, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (see, e.g., Abrams et al., Development 117:29 (1993); herein incorporated by reference in its entirety) and cis-parinaric acid, sensitive to the lipid peroxidation that accompanies apoptosis (see, e.g., Hockenbery et al., Cell 75:241 (1993); herein incorporated by reference in its entirety). It should be noted that the peptide and the fluorescent dyes are merely exemplary. It is contemplated that any peptide that effectively acts as a substrate for a caspase produced as a result of apoptosis finds use with the present invention.

In some embodiments, conjugation between a dendrimer (e.g., a terminal arm of a dendrimer) and a functional ligand is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino) pyridine) (see, e.g., International Patent Application No. PCT/US2010/042556, herein incorporated by reference in its entirety).

Functionalized nanoparticles (e.g., dendrimers) often contain moieties (including but not limited to ligands, functional ligands, conjugates, therapeutic agents, targeting agents, imaging agents, fluorophores) that are conjugated to the periphery. Such moieties may for example be conjugated to one or more dendrimer branch termini. Classical multi-step conjugation strategies used during the synthesis of functionalized dendrimers generate a stochastic distribution of products with differing numbers of ligands attached per dendrimer molecule, thereby creating a population of dendrimers with a wide distribution in the numbers of ligands attached. The low structural uniformity of such dendrimer populations negatively affects properties such as therapeutic potency, pharmacokinetics, or effectiveness for multivalent targeting. Difficulties in quantifying and resolving such populations to yield samples with sufficient structural uniformity can pose challenges. However, in some embodiments, use of separation methods (e.g., reverse phase chromatography) customized for optimal separation of dendrimer populations in conjunction with peak fitting analysis methods allows isolation and identification of subpopulations of functionalized dendrimers with high structural uniformity (see, e.g., U.S. Provisional Pat. App. No. 61/237,172; herein incorporated by reference in its entirety). In certain embodiments, such methods and systems provide a dendrimer product made by the process comprising: a) conjugation of at least one ligand type to a dendrimer to yield a population of ligand-conjugated dendrimers; b) separation of the population of ligand-conjugated dendrimers with reverse phase HPLC to result in subpopulations of ligand-conjugated dendrimers indicated by a chromatographic trace; and c) application of peak fitting analysis to the chromatographic trace to identify subpopulations of ligand-conjugated dendrimers wherein the structural uniformity of ligand conjugates per molecule of dendrimer within said subpopulation is, e.g., approximately 80% or more.

The present invention is not limited by the type of therapeutic agent delivered via multifunctional dendrimers of the present invention. For example, a therapeutic agent may be any agent selected from the group comprising, but not limited to, a pain relief agent, a pain relief agent antagonist, a chemotherapeutic agent, an anti-oncogenic agent, an anti-angiogenic agent, a tumor suppressor agent, an anti-microbial agent, or an expression construct comprising a nucleic acid encoding a therapeutic protein.

Indeed, in some embodiments of the present invention, methods and compositions are provided for the treatment of inflammatory diseases (e.g., dendrimers conjugated with therapeutic agents configured for treating inflammatory diseases). Inflammatory diseases include but are not limited to arthritis, rheumatoid arthritis, psoriatic arthritis, osteoarthritis, degenerative arthritis, polymyalgia rheumatic, ankylosing spondylitis, reactive arthritis, gout, pseudogout, inflammatory joint disease, systemic lupus erythematosus, polymyositis, and fibromyalgia. Additional types of arthritis include achilles tendinitis, achondroplasia, acromegalic arthropathy, adhesive capsulitis, adult onset Still's disease, anserine bursitis, avascular necrosis, Behcet's syndrome, bicipital tendinitis, Blount's disease, brucellar spondylitis, bursitis, calcaneal bursitis, calcium pyrophosphate dihydrate deposition disease (CPPD), crystal deposition disease, Caplan's syndrome, carpal tunnel syndrome, chondrocalcinosis, chondromalacia patellae, chronic synovitis, chronic recurrent multifocal osteomyelitis, Churg-Strauss syndrome, Cogan's syndrome, corticosteroid-induced osteoporosis, costosternal syndrome, CREST syndrome, cryoglobulinemia, degenerative joint disease, dermatomyositis, diabetic finger sclerosis, diffuse idiopathic skeletal hyperostosis (DISH), discitis, discoid lupus erythematosus, drug-induced lupus, Duchenne's muscular dystrophy, Dupuytren's contracture, Ehlers-Danlos syndrome, enteropathic arthritis, epicondylitis, erosive inflammatory osteoarthritis, exercise-induced compartment syndrome, Fabry's disease, familial Mediterranean fever, Farber's lipogranulomatosis, Felty's syndrome, Fifth's disease, flat feet, foreign body synovitis, Freiberg's disease, fungal arthritis, Gaucher's disease, giant cell arteritis, gonococcal arthritis, Goodpasture's syndrome, granulomatous arteritis, hemarthrosis, hemochromatosis, Henoch-Schonlein purpura, Hepatitis B surface antigen disease, hip dysplasia, Hurler syndrome, hypermobility syndrome, hypersensitivity vasculitis, hypertrophic osteoarthropathy, immune complex disease, impingement syndrome, Jaccoud's arthropathy, juvenile ankylosing spondylitis, juvenile dermatomyositis, juvenile rheumatoid arthritis, Kawasaki disease, Kienbock's disease, Legg-Calve-Perthes disease, Lesch-Nyhan syndrome, linear scleroderma, lipoid dermatoarthritis, Lofgren's syndrome, Lyme disease, malignant synovioma, Marfan's syndrome, medial plica syndrome, metastatic carcinomatous arthritis, mixed connective tissue disease (MCTD), mixed cryoglobulinemia, mucopolysaccharidosis, multicentric reticulohistiocytosis, multiple epiphyseal dysplasia, mycoplasmal arthritis, myofascial pain syndrome, neonatal lupus, neuropathic arthropathy, nodular panniculitis, ochronosis, olecranon bursitis, Osgood-Schlatter's disease, osteoarthritis, osteochondromatosis, osteogenesis imperfecta, osteomalacia, osteomyelitis, osteonecrosis, osteoporosis, overlap syndrome, pachydermoperiostosis Paget's disease of bone, palindromic rheumatism, patellofemoral pain syndrome, Pellegrini-Stieda syndrome, pigmented villonodular synovitis, piriformis syndrome, plantar fasciitis, polyarteritis nodos, Polymyalgia rheumatic, polymyositis, popliteal cysts, posterior tibial tendinitis, Pott's disease, prepatellar bursitis, prosthetic joint infection, pseudoxanthoma elasticum, psoriatic arthritis, Raynaud's phenomenon, reactive arthritis/Reiter's syndrome, reflex sympathetic dystrophy syndrome, relapsing polychondritis, retrocalcaneal bursitis, rheumatic fever, rheumatoid vasculitis, rotator cuff tendinitis, sacroiliitis, salmonella osteomyelitis, sarcoidosis, saturnine gout, Scheuermann's osteochondritis, scleroderma, septic arthritis, seronegative arthritis, shigella arthritis, shoulder-hand syndrome, sickle cell arthropathy, Sjogren's syndrome, slipped capital femoral epiphysis, spinal stenosis, spondylolysis, staphylococcus arthritis, Stickler syndrome, subacute cutaneous lupus, Sweet's syndrome, Sydenham's chorea, syphilitic arthritis, systemic lupus erythematosus (SLE), Takayasu's arteritis, tarsal tunnel syndrome, tennis elbow, Tietse's syndrome, transient osteoporosis, traumatic arthritis, trochanteric bursitis, tuberculosis arthritis, arthritis of Ulcerative colitis, undifferentiated connective tissue syndrome (UCTS), urticarial vasculitis, viral arthritis, Wegener's granulomatosis, Whipple's disease, Wilson's disease, and yersinial arthritis.

In some embodiments, the conjugated dendrimers of the present invention configured for treating autoimmune disorders and/or inflammatory disorders (e.g., rheumatoid arthritis) are co-administered to a subject (e.g., a human suffering from an autoimmune disorder and/or an inflammatory disorder) a therapeutic agent configured for treating autoimmune disorders and/or inflammatory disorders (e.g., rheumatoid arthritis). Examples of such agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), and glucocorticoids (e.g., prednisone, methylprednisone).

In some embodiments, the medical condition and/or disease is pain (e.g., chronic pain, mild pain, recurring pain, severe pain, etc.). In some embodiments, the conjugated dendrimers of the present invention are configured to deliver pain relief agents to a subject. In some embodiments, the dendrimer conjugates are configured to deliver pain relief agents and pain relief agent antagonists to counter the side effects of pain relief agents. The dendrimer conjugates are not limited to treating a particular type of pain and/or pain resulting from a disease. Examples include, but are not limited to, pain resulting from trauma (e.g., trauma experienced on a battlefield, trauma experienced in an accident (e.g., car accident)). In some embodiments, the dendrimer conjugates of the present invention are configured such that they are readily cleared from the subject (e.g., so that there is little to no detectable toxicity at efficacious doses).

In some embodiments, the disease is cancer. The present invention is not limited by the type of cancer treated using the compositions and methods of the present invention. Indeed, a variety of cancer can be treated including, but not limited to, prostate cancer, colon cancer, breast cancer, lung cancer and epithelial cancer.

In some embodiments, the disease is a neoplastic disease, selected from, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

In some embodiments, the composition is co-administered with an anti-cancer agent (e.g., Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda). Other anti-cancer agents include, but are not limited to, Antiproliferative agents (e.g., Piritrexim Isothionate), Antiprostatic hypertrophy agent (e.g., Sitogluside), Benign prostatic hyperplasia therapy agents (e.g., Tamsulosin Hydrochloride), Prostate growth inhibitor agents (e.g., Pentomone), and Radioactive agents: Fibrinogen 1 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; and Triolein I 131).

Additional anti-cancer agents include, but are not limited to anti-cancer Supplementary Potentiating Agents: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. Still other anticancer agents include, but are not limited to, annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO₄; mitoxantrone; mitozolomide; Pentostatin; and Tomudex. One particularly preferred class of anticancer agents are taxanes (e.g., paclitaxel and docetaxel). Another important category of anticancer agent is annonaceous acetogenin.

In some embodiments, the composition is co-administered with a pain relief agent. In some embodiments, the pain relief agents include, but are not limited to, analgesic drugs, anxiolytic drugs, anesthetic drugs, antipsychotic drugs, hypnotic drugs, sedative drugs, and muscle relaxant drugs.

In some embodiments, the analgesic drugs include, but are not limited to, non-steroidal anti-inflammatory drugs, COX-2 inhibitors, and opiates. In some embodiments, the non-steroidal anti-inflammatory drugs are selected from the group consisting of Acetylsalicylic acid (Aspirin), Amoxiprin, Benorylate/Benorilate, Choline magnesium salicylate, Diflunisal, Ethenzamide, Faislamine, Methyl salicylate, Magnesium salicylate, Salicyl salicylate, Salicylamide, arylalkanoic acids, Diclofenac, Aceclofenac, Acemethacin, Alclofenac, Bromfenac, Etodolac, Indometacin, Nabumetone, Oxametacin, Proglumetacin, Sulindac, Tolmetin, 2-arylpropionic acids, Ibuprofen, Alminoprofen, Benoxaprofen, Carprofen, Dexibuprofen, Dexketoprofen, Fenbufen, Fenoprofen, Flunoxaprofen, Flurbiprofen, Ibuproxam, Indoprofen, Ketoprofen, Ketorolac, Loxoprofen, Naproxen, Oxaprozin, Pirprofen, Suprofen, Tiaprofenic acid), N-arylanthranilic acids, Mefenamic acid, Flufenamic acid, Meclofenamic acid, Tolfenamic acid, pyrazolidine derivatives, Phenylbutazone, Ampyrone, Azapropazone, Clofezone, Kebuzone, Metamizole, Mofebutazone, Oxyphenbutazone, Phenazone, Sulfinpyrazone, oxicams, Piroxicam, Droxicam, Lornoxicam, Meloxicam, Tenoxicam, sulphonanilides, nimesulide, licofelone, and omega-3 fatty acids. In some embodiments, the COX-2 inhibitors are selected from the group consisting of Celecoxib, Etoricoxib, Lumiracoxib, Parecoxib, Rofecoxib, and Valdecoxib. In some embodiments, the opiate drugs are selected from the group consisting of natural opiates, alkaloids, morphine, codeine, thebaine, semi-synthetic opiates, hydromorphone, hydrocodone, oxycodone, oxymorphone, desomorphine, diacetylmorphine (Heroin), nicomorphine, dipropanoylmorphine, diamorphine, benzylmorphine, Buprenorphine, Nalbuphine, Pentazocine, meperidine, diamorphine, ethylmorphine, fully synthetic opioids, fentanyl, pethidine, Oxycodone, Oxymorphone, methadone, tramadol, Butorphanol, Levorphanol, propoxyphene, endogenous opioid peptides, endorphins, enkephalins, dynorphins, and endomorphins.

In some embodiments, the anxiolytic drugs include, but are not limited to, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze, Triazolam, serotonin lA agonists, Buspirone (BuSpar), barbituates, amobarbital (Amytal), pentobarbital (Nembutal), secobarbital (Seconal), Phenobarbital, Methohexital, Thiopental, Methylphenobarbital, Metharbital, Barbexaclone), hydroxyzine, cannabidiol, valerian, kava (Kava Kava), chamomile, Kratom, Blue Lotus extracts, Sceletium tortuosum (kanna) and bacopa monniera.

In some embodiments, the anesthetic drugs include, but are not limited to, local anesthetics, procaine, amethocaine, cocaine, lidocaine, prilocaine, bupivacaine, levobupivacaine, ropivacaine, dibucaine, inhaled anesthetics, Desflurane, Enflurane, Halothane, Isoflurane, Nitrous oxide, Sevoflurane, Xenon, intravenous anesthetics, Barbiturates, amobarbital (Amytal), pentobarbital (Nembutal), secobarbital (Seconal), Phenobarbital, Methohexital, Thiopental, Methylphenobarbital, Metharbital, Barbexaclone)), Benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, Etomidate, Ketamine, and Propofol.

In some embodiments, the antipsychotic drugs include, but are not limited to, butyrophenones, haloperidol, phenothiazines, Chlorpromazine (Thorazine), Fluphenazine (Prolixin), Perphenazine (Trilafon), Prochlorperazine (Compazine), Thioridazine (Mellaril), Trifluoperazine (Stelazine), Mesoridazine, Promazine, Triflupromazine (Vesprin), Levomepromazine (Nozinan), Promethazine (Phenergan)), thioxanthenes, Chlorprothixene, Flupenthixol (Depixol and Fluanxol), Thiothixene (Navane), Zuclopenthixol (Clopixol & Acuphase)), clozapine, olanzapine, Risperidone (Risperdal), Quetiapine (Seroquel), Ziprasidone (Geodon), Amisulpride (Solian), Paliperidone (Invega), dopamine, bifeprunox, norclozapine (ACP-104), Aripiprazole (Abilify), Tetrabenazine, and Cannabidiol.

In some embodiments, the hypnotic drugs include, but are not limited to, Barbiturates, Opioids, benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, nonbenzodiazepines, Zolpidem, Zaleplon, Zopiclone, Eszopiclone, antihistamines, Diphenhydramine, Doxylamine, Hydroxyzine, Promethazine, gamma-hydroxybutyric acid (Xyrem), Glutethimide, Chloral hydrate, Ethchlorvynol, Levomepromazine, Chlormethiazole, Melatonin, and Alcohol.

In some embodiments, the sedative drugs include, but are not limited to, barbituates, amobarbital (Amytal), pentobarbital (Nembutal), secobarbital (Seconal), Phenobarbital, Methohexital, Thiopental, Methylphenobarbital, Metharbital, Barbexaclone), benzodiazepines, alprazolam, bromazepam (Lexotan), chlordiazepoxide (Librium), Clobazam, Clonazepam, Clorazepate, Diazepam, Midazolam, Lorazepam, Nitrazepam, temazepam, nimetazepam, Estazolam, Flunitrazepam, oxazepam (Serax), temazepam (Restoril, Normison, Planum, Tenox, and Temaze), Triazolam, herbal sedatives, ashwagandha, catnip, kava (Piper methysticum), mandrake, marijuana, valerian, solvent sedatives, chloral hydrate (Noctec), diethyl ether (Ether), ethyl alcohol (alcoholic beverage), methyl trichloride (Chloroform), nonbenzodiazepine sedatives, eszopiclone (Lunesta), zaleplon (Sonata), zolpidem (Ambien), zopiclone (Imovane, Zimovane)), clomethiazole (clomethiazole), gamma-hydroxybutyrate (GHB), Thalidomide, ethchlorvynol (Placidyl), glutethimide (Doriden), ketamine (Ketalar, Ketaset), methaqualone (Sopor, Quaalude), methyprylon (Noludar), and ramelteon (Rozerem).

In some embodiments, the muscle relaxant drugs include, but are not limited to, depolarizing muscle relaxants, Succinylcholine, short acting non-depolarizing muscle relaxants, Mivacurium, Rapacuronium, intermediate acting non-depolarizing muscle relaxants, Atracurium, Cisatracurium, Rocuronium, Vecuronium, long acting non-depolarizing muscle relaxants, Alcuronium, Doxacurium, Gallamine, Metocurine, Pancuronium, Pipecuronium, and d-Tubocurarine.

In some embodiments, the composition is co-administered with a pain relief agent antagonist. In some embodiments, the pain relief agent antagonists include drugs that counter the effect of a pain relief agent (e.g., an anesthetic antagonist, an analgesic antagonist, a mood stabilizer antagonist, a psycholeptic drug antagonist, a psychoanaleptic drug antagonist, a sedative drug antagonist, a muscle relaxant drug antagonist, and a hypnotic drug antagonist). In some embodiments, pain relief agent antagonists include, but are not limited to, a respiratory stimulant, Doxapram, BIMU-8, CX-546, an opiod receptor antagonist, Naloxone, naltrexone, nalorphine, levallorphan, cyprodime, naltrindole, norbinaltorphimine, buprenorphine, a benzodiazepine antagonist, flumazenil, a non-depolarizing muscle relaxant antagonist, and neostigmine.

Where clinical applications are contemplated, in some embodiments of the present invention, the dendrimer conjugates are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In preferred embodiments, the dendrimer conjugates are used in conjunction with appropriate salts and buffers to render delivery of the compositions in a stable manner to allow for uptake by target cells. Buffers also are employed when the dendrimer conjugates are introduced into a patient. Aqueous compositions comprise an effective amount of the dendrimer conjugates to cells dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with vectors, cells, or tissues, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active dendrimer conjugates may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In some embodiments, a therapeutic agent is released from dendrimer conjugates within a target cell (e.g., within an endosome). This type of intracellular release (e.g., endosomal disruption of a linker-therapeutic conjugate) is contemplated to provide additional specificity for the compositions and methods of the present invention. The present invention provides dendrimers with multiple (e.g., 100-150) reactive sites for the conjugation of linkers and/or functional groups comprising, but not limited to, therapeutic agents, targeting agents, imaging agents and biological monitoring agents.

The compositions and methods of the present invention are contemplated to be equally effective whether or not the dendrimer conjugates of the present invention comprise a fluorescein (e.g. FITC) imaging agent. Thus, each functional group present in a dendrimer composition is able to work independently of the other functional groups. Thus, the present invention provides dendrimer conjugates that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, dendrimer conjugates are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The dendrimer conjugates also may be formulated as inhalants for the treatment of lung cancer and such like.

In some embodiments, the present invention also provides kits comprising one or more of the reagents and tools necessary to generate conjugate an azide-derivatized functional group with an alkyne-derivatized dendrimer within a biological setting.

### EXAMPLES

The following examples are provided in order to demonstrate and further illustrate certain preferred embodiments and aspects of the present invention and are not to be construed as limiting the scope thereof.

### Example 1

Previous experiments involving dendrimer related technologies are located in U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023, each herein incorporated by reference in their entireties.

### Example 2

This example describes the materials and methods for Examples 3 and 4.

### Materials

All solvents and chemicals were of reagent grade quality, purchased from Sigma-Aldrich Chemical Co. and used without further purification unless otherwise noted. Folic acid (FA), folate binding protein extracted from bovine milk (FBP), acetic anhydride, ethylenediamine, methanol, dimethylsulfoxide (DMSO), penicillin/streptomycin, and fetal bovine serum were purchased from Sigma-Aldrich (St. Louis, Mo.). AlexaFluor 488 carboxylic acid linked by succinimidyl ester (AF488), Trypsin-EDTA, Dulbecco's PBS, and RPMI 1640 (with and without folic acid) were supplied by Invitrogen (Gaithersburg, Md.).

### General Information

¹H NMR spectra were obtained using a Varian Inova 500 MHz spectrometer. Matrix-assisted laser desorption ionization time-of-flight mass spectra (MALDI-TOF-MS) were recorded on a PE Biosystems Voyager System 6050, using 2,5-dihydroxybenzoic acid (DHB) as the matrix.

All the conjugates and their intermediate reaction products were analyzed by MALDI-TOF-MS, HPLC, and NMR. The number of ligands that attached to the dendrimer was obtained from the integration of the methyl protons of the terminal acetyl groups to the aromatic protons on the conjugated ligands (e.g., FA and MTX) (see, e.g., Mullen, D. G. et al. Acs Nano 4, 657-670; herein incorporated by reference in its entirety). The number of acetyl groups per dendrimer was determined by first computing the total number of end groups from the number average molecular weight from gel permeation chromatography (GPC) and potentiometric titration data for G5-NH₂(100%) as previously described (see, e.g., Majoros, I. J., et al., Journal of Medicinal Chemistry 48, 5892-5899; herein incorporated by reference in its entirety). The total number of end groups was applied to the ratio of primary amines to acetyl groups, obtained from the ¹H NMR of the partially acetylated dendrimer, to compute the average number of acetyl groups per dendrimer.

### Synthesis

3-azido-7-hydroxy coumarin was synthesized according to the literature (see, e.g., Sivakumar, K. et al. Org Lett 6, 4603-4606; herein incorporated by reference in its entirety). G5-NHAc₈₀-Alkyne₁₂ 1 and Alkyne-terminated dendrimer G5-NHAc₈₀-Alkyne₈-FA₄ 2 were prepared as reported (see, e.g., Hong, et al. Chem Biol 14, 107-115; herein incorporated by reference in its entirety). Synthesis of G5-NHAc₈₀-Alkyne₈-FA₄-Gly 3. G5-NHAc₈₀-Alkyne₈-FA₄ 2 (3.3 mg, 0.10 μmol) was dissolved in 200 μl DMSO. Large excess glycidol (1 mg) was added (>10eq. per NH2). The reaction mixture was stirred at room temperature overnight. Sample was purified using 10,000 MWCO centrifugal filtration devices. Purification consisted of ten cycles (20 min at 4800 rpm) using PBS (5 cycles) and DI water (5 cycles). The purified dendrimer samples were lyophilized to yield 3 as yellow solid (2.9 mg, 83%).

Synthesis of G5-NHAc₈₀-Alkyne₁₂-Mannose₁₅-Gly 5. α-D-Mannopyranosylphenyl isothiocyanate (0.37 mg, 1.2 μmol) in 100 μl DMSO was added to a solution of G5-NHAc80-Alkyne12 1 (2.6 mg, 0.08 μmol) and N,N-Diisopropylethylamine (DIPEA) (0.3 mg, 0.16 μmol) in 150 μl DMSO. The reaction was stirred at room temperature for 24 hours. Large excess glycidol (1 mg) was added (>10eq. per NH₂). The reaction mixture was stirred at room temperature overnight. Sample was purified using 10,000 MWCO centrifugal filtration devices. Purification consisted of ten cycles (20 min at 4800 rpm) using PBS (5 cycles) and DI water (5 cycles). The purified dendrimer samples were lyophilized to yield 5 as white solid (1.6 mg, 55%).

Synthesis of G5-NHAc₈₀-Alkyne₁₂-Gly 6. G5-NHAc₈₀-Alkyne₁₂ 1 (3.6 mg, 0.11 μmol) was dissolved in 300 μl DMSO. Large excess glycidol (1 mg) was added (>10eq. per NH₂). The reaction mixture was stirred at room temperature overnight. Sample was purified using 10,000 MWCO centrifugal filtration devices. Purification consisted of ten cycles (20 min at 4800 rpm) using PBS (5 cycles) and DI water (5 cycles). The purified dendrimer samples were lyophilized to yield 6 as white solid (3.7 mg, 94%).

### Characterization of G5 PAMAM Dendrimer Conjugates

¹H NMR spectra were taken in D₂O and were used to provide integration values for structural analysis using a Bruker AVANCE DRX 500 instrument. Ultra performance liquid chromatography (UPLC) analysis was carried out on a Waters Acquity Peptide Mapping System equipped with a Waters photodiode array detector, a column manager that facilitates 4 column housing, and a sample manager. The instrument is controlled by Empower 2 software. The analysis was carried out using a gradient elution beginning with 99:1 (v/v) water/acetonitrile (ACN) reaching 20:80 water/ACN in 13.40 minutes. Flow rate was maintained at 0.208 mL/min and trifluoroacetic acid (TFA) at 0.14 wt % concentration was added in water as well as in ACN as a counter ion. The column temperature was maintained at 35° C.

### Cell Culture and Flow Cytometry Measurements

The KB cell line was purchased from the American Type Tissue Collection (ATCC, Manassas, Va.) and grown continuously as a monolayer at 37° C. and 5% CO₂ in RPMI 1640 medium (Mediatech, Herndon, Va.). The RPMI 1640 medium was supplemented with penicillin (100 units/ml), streptomycin (100 mg/ml), and 10% heat-inactivated, low endotoxin fetal bovine serum (FBS) before use (Mediatech). KB cells were cultured in RPMI 1640 medium without folic acid for at least 4 days before experiments, resulting in the folic acid receptor overexpressing KB (FAR-KB) cell line. Bone marrow derived macrophages (BMDM) were obtained as previously described. Briefly, bone marrow precursors from female C57BL/6 mice were isolated and propagated for 7 days in RPMI 1640 supplemented with penicillin (100 units/ml), streptomycin (100 mg/ml), and 10% heat inactivated low endotoxin fetal bovine serum with L-conditioned media. Following propagation, BMDMs were polarized with murine recombinant proteins M-CSF (10 ng/ml), IL4 (50 ng/ml), IL10 (10 ng/ml), or GM-CSF (10 ng/ml) for 72 hours (Peprotech). For the flow cytometry measurements, cells were seeded on a 12-well plate for tissue culture at a concentration of 23105 cells/well and grown in folic acid deficient RPMI 1640 media (Mediatech, Herndon, Va.) at 37° C., 5% CO2 for 24 hr. The cells were then incubated with the series of the prepared nanodevices at either 37° C. for 1 hr. After removal of supernatants, cells were trypsinized and collected into FACS tubes, followed by centrifugation at 1500 rpm for 5 min to obtain cell pellets. The pellets were washed with PBS twice using a repetitive centrifugation and resuspension process and then finally resuspended in PBS with 0.1% bovine serum albumin. The FACS sample preparation was performed on ice to inhibit cellular reactions such as further uptake. Fluorescence signal intensities from the samples were measured using a Coulter EPICS/XL MCL Beckman-Coulter flow cytometer, and data were analyzed using FloJo 8.2 software (TreeStar).

### Confocal Laser Scanning Microscopy (CLSM) Observation

FAR-KB cells were seeded on a glass bottomed Petri dish. The cell culture medium was replaced by 2 ml of each dendrimer nanodevice solution in PBS, followed by incubation at 37° C. under 5% CO₂ for 1 hr. The nanodevice-containing solution was removed, and the resulting cell monolayer was washed with PBS at least three times. Cells were fixed with 4% formaldehyde in PBS at room temperature for 15 min and then washed twice with PBS. Cells were stained by DAPI and conserved in an anti-photobleaching agent. Confocal images were then taken on an Olympus FV-500 confocal microscope using a 1003, 1.4 NA oil immersion objective. To visualize AlexaFluor 488 attached on dendrimers, the 488 nm line of an argon ion laser was used for excitation, and the emission was filtered at 505-525 IF nm. To visualize AlexaFluor 555 attached on dendrimers, the 543 nm line of an HeNe laser was used for excitation, and the emission was filtered at 555-625 IF nm.

### Detection of Dendrimer Particles In Situ

After 4% paraformaldehyde fixation and permeabilization with 10% saponin in PBS and BSA, cells were rinsed and then stained by incubating cells for 30 min with 100 mM Tris (pH 8.5), 1 mM CuSO₄, 1-100 μM fluorescent azide (in DMSO), and 100 mM ascorbic acid (added last to the mix in 0.5 M stock in water). The staining solution was prepared fresh each time and was used for staining cells immediately after the addition of ascorbate. After staining, cells were washed with 10% saponin solution and assessed by flow cytometry.

### Example 3

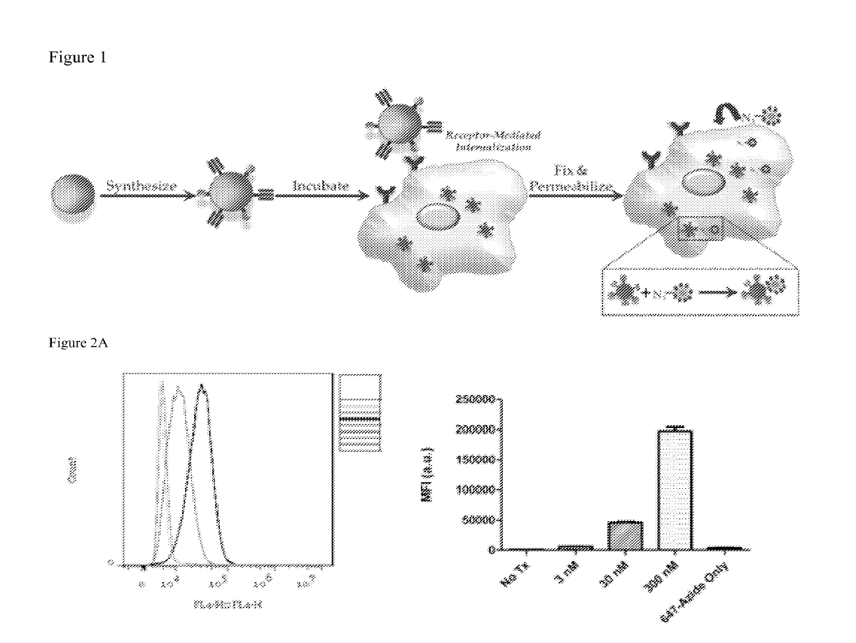

FIG. 1 summarizes the general components of a bioorthogonal chemical reporter embodiment of the present invention. To demonstrate the feasibility of this approach, generation 5 (G5) poly(amidoamine) (PAMAM) dendrimers functionalized with alkyne moieties were synthesized. The dendrimer scaffolds had approximately 12 alkyne handles as determined by NMR and MALDI-TOF mass analyses. Some of these macromolecules were further modified with folic acid (FA) as a targeting moiety. To confirm that the coupling efficiencies of different dendrimer conjugates were similar, a “click” coumarin-fluorescence reporter assay was used to monitor the efficiency of the CuAAC reaction using different dendrimer conjugates. The covalent coupling of a fluorescently silent 3-azido-7-hydroxy coumarin reporter with the dendrimer alkyne linker produces a strongly fluorescent triazole-linked conjugate (see, e.g., Sivakumar, K. et al., Org Lett 6, 4603-4606 (2007); herein incorporated by reference in its entirety). To evaluate reaction efficiency, a 3-azido-7-hydroxy coumarin fluorescent reporter was coupled to different alkyne-functionalized dendrimers and the changes in fluorescent intensity monitored. No significant differences in coupling efficiency between the different dendrimer conjugates used in this study were noted.

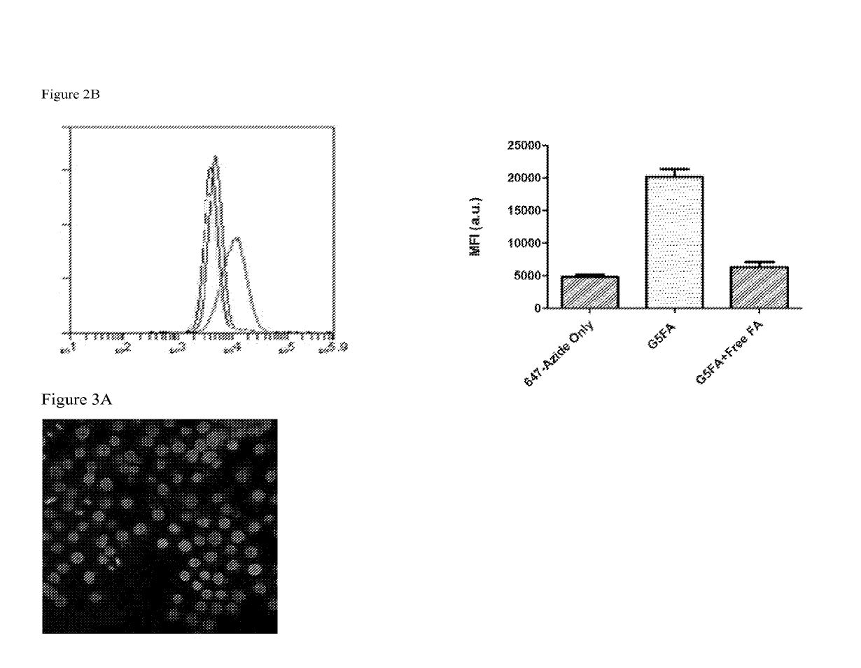

FA functionalized nanoparticles are internalized by receptor-mediated endocytosis through the folate receptor alpha (FRα) in KB cells and folate receptor 13 (FR(3) in activated macrophages (see, e.g., Kukowska-Latallo, J. F. et al. Cancer Res 65, 5317-5324 (2005); Thomas, T. P. et al. J Med Chem 48, 3729-3735 (2005); Xia, W. et al. Blood 113, 438-446 (2009); Puig-Kroger, A. et al. Cancer Res 69, 9395-9403 (2009); each herein incorporated by reference in their entireties). In FRα over-expressing KB cells, FA-dendrimer alkyne nanodevices were incubated for 1 hour at 37° C. Following incubation, cells were washed, fixed, and then permeabilized prior to performing the in situ CuAAC reaction using an azide flourophore to detect the internalized dendrimer nanoparticles. FA-dendrimer alkyne conjugates demonstrated dose-dependent cellular uptake similar to that of traditional FA-dendrimer conjugates (FIG. 2). The non-targeted dendrimer conjugates did not demonstrate any significant uptake compared to KB cells stained with the azide-fluorophore alone. The uptake of the FA, alkyne substituted dendrimers could be inhibited by pretreatment of KB cells with 100 μM FA, further demonstrating the specificity of these targeted nanodevices (FIG. 2). Another advantage of this reporter system is that the amount of fluorescent signal can be tuned by varying the concentration and reaction times of fluorescent azide reporters providing more downstream flexibility to use other fluorescent markers in parallel.

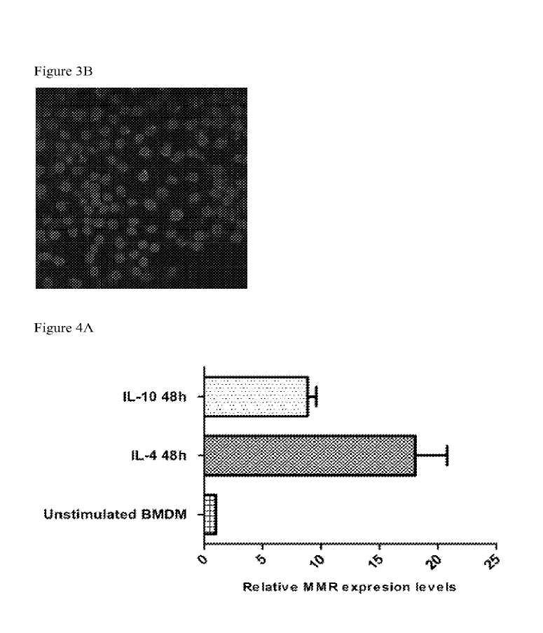

To assess the utility of this reporter strategy for fluorescent imaging applications and to demonstrate the flexibility afforded by the in situ labeling of nanoparticles, used different azide flourophores to monitor conjugate uptake in KB cells over-expressing FRα was studied. KB cells were incubated with FA-dendrimer alkyne conjugates for 1 hour at 37° C. Following incubation, cells were washed, fixed, and then permeabilized before covalently attaching AlexaFluor 488 and 555 azide reporters using the CuAAC reaction (FIG. 3). Detection of the nanoparticle scaffold with diverse azide-fluorophores was efficient and effective for microscopy applications and provided us with the capability to stain with several fluorescent probes without resynthesizing dendrimer conjugates.

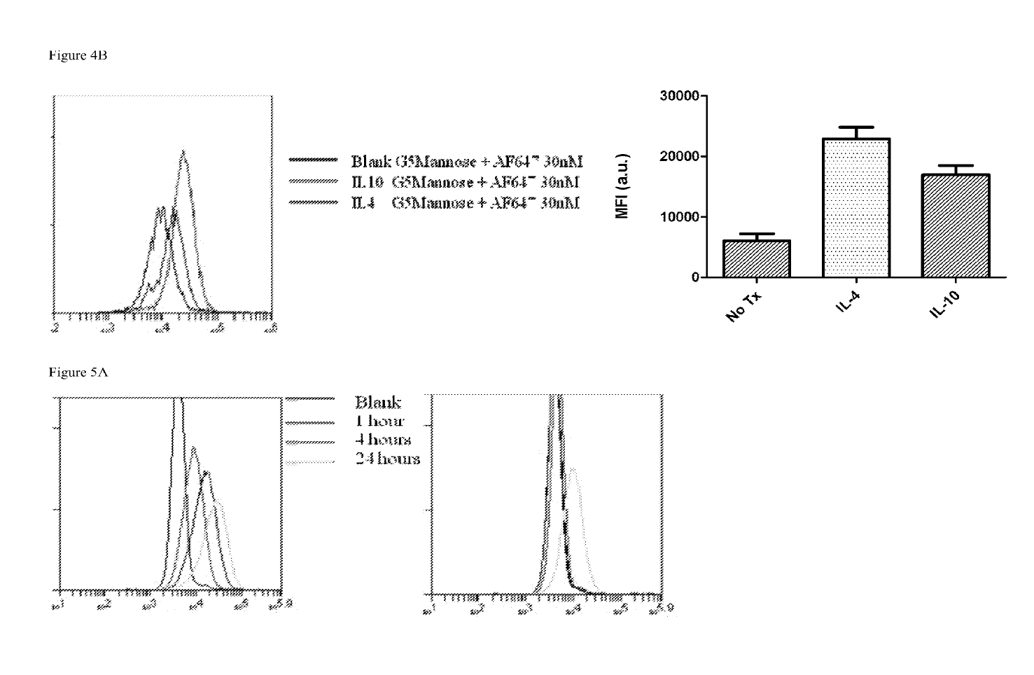

To demonstrate the broad applicability of this alkyne reporter strategy using a different receptor-ligand system, G5 PAMAM dendrimer alkyne nanoparticles functionalized with the carbohydrate mannose as the targeting moiety were synthesized. Mannose is a high-affinity ligand for the macrophage mannose receptor (MMR/CD206), a type 1 membrane immune receptor that mediates endocytosis of distinct glycoproteins (see, e.g., Wileman, T. E., et al., Proc Natl Acad Sci USA 83, 2501-2505 (1986); herein incorporated by reference in its entirety). The MMR is upregulated in bone marrow derived macrophages (BMDM) under specific polarizing conditions providing an inducible system to validate the targeting of the mannose targeted dendrimer conjugates. BMDM were generated as previously reported and differentiated with cytokines (IL-4 and IL-10) known to upregulate the MMR in macrophages (see, e.g., Biswas, S. K. & Mantovani, A. Nat Immunol 11, 889-896 (2009); Saccani, A. et al. Cancer Res 66, 11432-11440 (2010); each herein incorporated by reference in their entireties). Following the differentiation of BMDMs, mannose-dendrimer conjugates were incubated with these primary cells for 6 hours at 37° C. The BMDM were then washed, fixed, and permeabilized prior to detection of the mannose-dendrimer conjugates. BMDMs differentiated under conditions that upregulate the mannose receptor (IL-4 and IL-10) demonstrated increased binding and uptake of mannose-targeted dendrimers compared to their non-targeted controls (FIG. 4). The uptake in BMDMs treated with mannose functionalized dendrimers is consistent with receptor-mediated endocytosis facilitated by the mannose targeting moiety and further demonstrates the generalizability of our bioorthogonal reporter to monitor diverse nanoparticles and receptor-ligand systems.

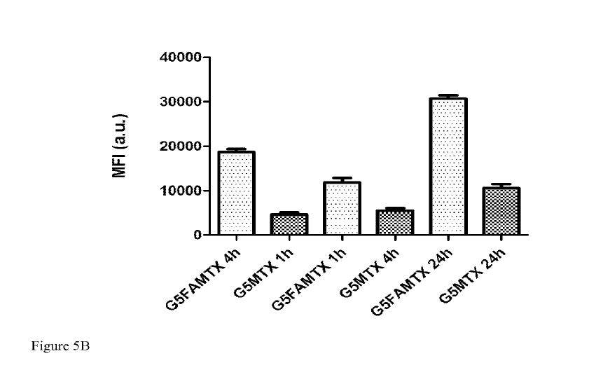

After confirming the efficacy of this reporter strategy, this approach was used to probe the biological properties of two therapeutic dendrimer conjugates derivatized with alkyne handles. The approach to preparing dendrimer conjugates utilized a triazine small molecule as a trifunctional scaffold on which two sites were used for conjugating either methotrexate (MTX) and/or FA while the third functional site was modified with a 3-azidopropan-1-amine linker for conjugation to the dendrimer backbone. These triazine derivatives were attached to an alkyne ligand-modified PAMAM dendrimer platform using click chemistry. Because of the modularity of this approach, the distributions between functionalized triazine rings and remaining alkyne linkers were similar between therapeutic dendrimer conjugates allowing the kinetics of internalization of distinct therapeutic conjugates without the confounding effects of varying distributions inherent to stepwise, polymer synthetic schemes to be studied. Methotrexate has been shown to bind both folate receptors α and β; however, in comparison to folic acid, methotrexate has a 100 fold lower affinity for the folate receptor (see, e.g., van der Heijden, J. W. et al. Arthritis Rheum 60, 12-21 (2006); herein incorporated by reference in its entirety). Nanoparticle platforms with multiple targeting moieties typically have greatly enhanced binding avidities compared with to their monomeric counterparts (see, e.g., Hong, S. et al. Chem Biol 14, 107-115 (2006); Tassa, C. et al. Bioconjug Chem 21, 14-19 (2007); each herein incorporated by reference in their entireties). In order to compare the internalization ability of different substituted dendrimers in a biological system, therapeutic dendrimer conjugates functionalized with either methotrexate or folate and methotrexate were incubated in FRα over-expressing KB cells for 1, 4, and 24 hours. Following incubation, cells were washed, fixed, and then permeabilized and the nanoparticle scaffolds were detected using our CuAAC reporter strategy. Both the FA and MTX targeted dendrimers demonstrated specific uptake in FRα overexpressing KB cells. Interestingly, the FA targeted methotrexate dendrimer conjugate had increased uptake at all the time points assessed when compared to the methotrexate dendrimer conjugate (FIG. 5). Although the results suggest that methotrexate dendrimer nanoparticles do internalize into FRα over-expressing KB cells, the internalization rate and total amounts are greatly reduced compared to their FA targeted conjugates. This CuAAC reporter strategy coupled with the modularity of the synthetic approach greatly expands the types of analyses that can be pursued to understand the complex interactions between nanoparticles and biological systems.

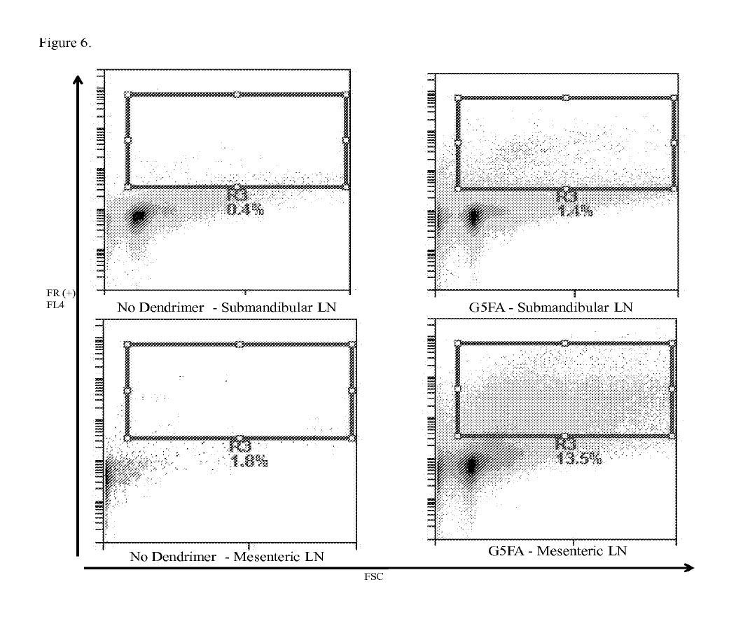

To further demonstrate the utility of our CuAAC reporter strategy, the ability to monitor the in vivo trafficking of FA-targeted dendrimer nanoparticles in a murine model of inflammation was investigated. On day 1, mice (n=2) were treated IP with thioglycollate to stimulate folate receptor positive macrophage infiltration into the peritoneal cavity (see, e.g., Xia, W. et al. Blood 113, 438-446 (2005); herein incorporated by reference in its entirety). On day 3, mice were administered folate targeted dendrimers IP (35 mg/kg). On day 4, peritoneal macrophages (PEM) as well as the spleen, submandibular lymph nodes, and mesenteric lymph nodes were isolated and processed as using standard techniques. Single cell suspensions were made and analyzed by flow cytometry for co-expression of the macrophage marker CD11b and the FA-dendrimer scaffolds. As expected, there was significant uptake of folate nanodevices detected in the PEM as well as in CD 11b⁽⁺⁾ cells in the meseneteric lymph nodes which are the primary lymph organs draining the peritoneal cavity (FIG. 6). Compared to the cervical lymph nodes (secondary lymph organs not draining the peritoneal cavity), there was a greater proportion of double-positive CD11b and FR cells in the mesenteric lymph nodes consistent with the trafficking of these activated macrophages. As expected, the peritoneal macrophages from mice stimulated with thioglycollate also had a much higher uptake of FA-dendrimer nanoparticles consistent with the high prevalence of FA(+) macrophages in the peritoneal cavity. These results suggest that, for example, click chemistry reporter strategy can be used to track nanoparticle dynamics with excellent sensitivity and specificity in animal models further expanding the utility of our approach to help understand the complex relationship between nanoparticles and biological systems.

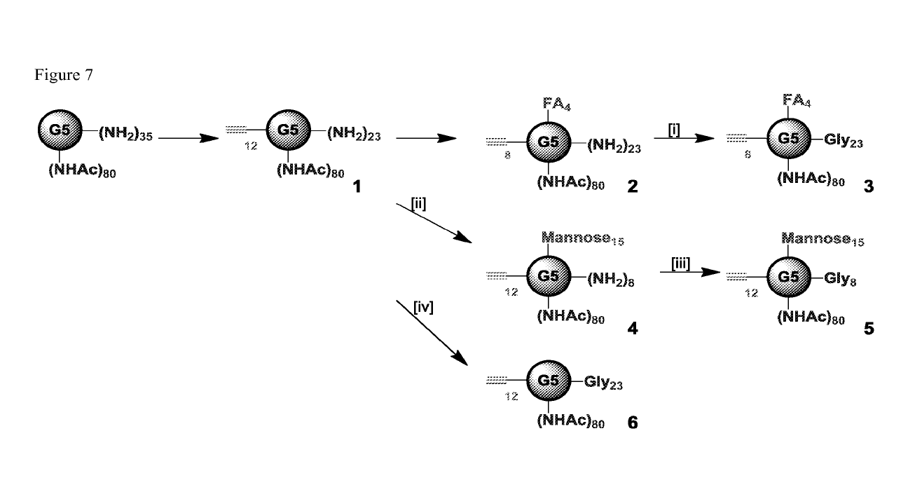

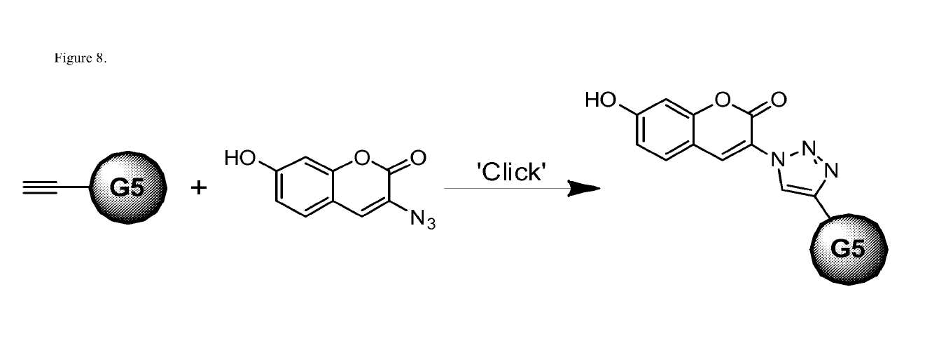

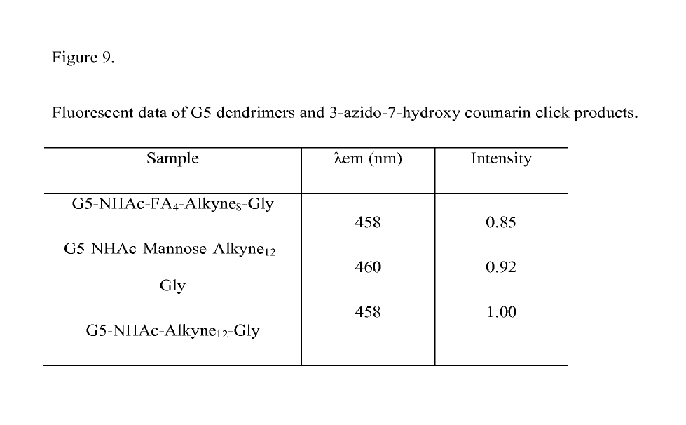

FIG. 7 shows a scheme for chemical synthesis of folate and mannose functionalized, dendrimer-alkyne conjugates. FIG. 8 shows a schematic illustration of click efficiency tests using 3-azido-7-hydroxy coumarin fluorescent assay. FIG. 9 presents a table describing click efficiency using 3-azido-7-hydroxy coumarin fluorescent assay.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in dendrimer synthesis, drug delivery, or related fields are intended to be within the scope of the following claims.

We claim:

1. A method for synthesizing a functionalized dendrimer within a biological sample, comprising administering to said biological sample one or more alkyne-derivatized dendrimers and one or more azide-derivatized functional groups such that upon contact within the biological sample the functional group conjugates with said dendrimer.

2. The method of claim 1, wherein said functional group conjugates with said dendrimer via a cycloaddition reaction.

3. The method of claim 2, wherein said cycloaddition reaction is a 1, 3 dipolar cycloaddition reaction.

4. The method of claim 1, wherein said alkyne-derivatized dendrimer is conjugated with one or more functional groups prior to administration to the biological sample.

5. The method of claim 4, wherein said functional group is selected from the group consisting of an imaging agent, a therapeutic agent, a targeting agent, and a trigger agent.

6. The method of claim 1, wherein said functional group is selected from the group consisting of an imaging agent, a therapeutic agent, a targeting agent, and a trigger agent.

7. The method of claim 1, wherein said biological sample is a mammalian biological sample.

8. A method for treating a disorder comprising administering to a subject having said disorder one or more alkyne-derivatized dendrimers conjugated with a targeting agent and one or more azide-derivatized therapeutic agents such that upon contact within said subject the therapeutic agent conjugates with said dendrimer.

9. The method of claim 8, wherein said alkyne-derivatized dendrimer is conjugated with one or more therapeutic agents prior to administration to the biological sample.

10. The method of claim 8, wherein said therapeutic agent conjugates with said dendrimer via a cycloaddition reaction.

11. The method of claim 10, wherein said cycloaddition reaction is a 1, 3 dipolar cycloaddition reaction.

12. A method for monitoring a functionalized dendrimer within a biological sample, comprising administering to the biological sample one or more alkyne-derivatized dendrimers and one or more azide-derivatized imaging agents such that upon contact within the biological sample the imaging agent conjugates with said dendrimer, and monitoring said imaging agent conjugated with said dendrimer.

13. The method of claim 12, wherein said imaging agent conjugates with said dendrimer via a cycloaddition reaction.

14. The method of claim 13, wherein said cycloaddition reaction is a 1, 3 dipolar cycloaddition reaction.
